(19) United States
(12) Patent Application Publication
(10) Pub. No.: US 2024/0189628 A1
(43) Pub. Date: Jun. 13, 2024

(54) ULTRASOUND COUPLING DEVICE FOR HISTOTRIPSY SYSTEMS AND METHODS

(71) Applicant: HistoSonics, Inc., Plymouth, MN (US)

(72) Inventors: Justin S. GRUMBIR, Minnetonka, MN (US); Franklin J. BURQUEST, Crystal, MN (US); Jon D. SCHELL, Shoreview, MN (US); Michael STORE, Hudson, WI (US); Marc T. MCCAULEY, Elk River, MN (US); Ellie L. JOHNSEN, Forest Lake, MN (US); Jonathan P. BOGOTT, Minnetonka, MN (US); Carole L. NORRIS, Saint Paul, MN (US)

(21) Appl. No.: 18/535,877

(22) Filed: Dec. 11, 2023

Related U.S. Application Data
(60) Provisional application No. 63/386,822, filed on Dec. 9, 2022.

(51) Int. Cl. A61N 7/00

(52) U.S. Cl. CPC A61N 7/00; A61N 2007/0039

(57) ABSTRACT

A histotripsy therapy system configured for the treatment of tissue is provided, which may include any number of features. Provided herein are systems and methods that provide efficacious non-invasive and minimally invasive therapeutic, diagnostic and research procedures. Additional embodiments herein provide coupling constraint configured to interface with a coupling assembly during a histotripsy procedure.

### PRIORITY CLAIM

This patent application claims priority to U.S. provisional patent application No. 63/386,822, titled “ULTRASOUND COUPLING DEVICE FOR HISTOTRIPSY SYSTEMS AND METHODS” and filed on Dec. 9, 2022, which is herein incorporated by reference in its entirety.

### INCORPORATION BY REFERENCE

All publications and patent applications mentioned in this specification are herein incorporated by reference to the same extent as if each individual publication or patent application was specifically and individually indicated to be incorporated by reference.

### FIELD

The present disclosure details novel high intensity therapeutic ultrasound (HITU) systems configured to produce acoustic cavitation, methods, devices and procedures for the minimally and non-invasive treatment of healthy, diseased and/or injured tissue. The acoustic cavitation systems and methods described herein, also referred to Histotripsy, may include transducers, drive electronics, positioning robotics, imaging systems, and integrated treatment planning and control software to provide comprehensive treatment and therapy for soft tissues in a patient.

### BACKGROUND

Histotripsy, or pulsed ultrasound cavitation therapy, is a technology where extremely short, intense bursts of acoustic energy induce controlled cavitation (microbubble formation) within the focal volume. The vigorous expansion and collapse of these microbubbles mechanically homogenizes cells and tissue structures within the focal volume. This is a very different end result than the coagulative necrosis characteristic of thermal ablation. To operate within a non-thermal, Histotripsy realm; it is necessary to deliver acoustic energy in the form of high amplitude acoustic pulses with low duty cycle.

Compared with conventional focused ultrasound technologies, Histotripsy has important advantages: 1) the destructive process at the focus is mechanical, not thermal; 2) cavitation appears bright on ultrasound imaging thereby confirming correct targeting and localization of treatment; 3) treated tissue generally, but not always, appears darker (more hypocchoic) on ultrasound imaging, so that the operator knows what has been treated; and 4) Histotripsy produces lesions in a controlled and precise manner. It is important to emphasize that unlike thermal ablative technologies such as microwave, radiofrequency, high-intensity focused ultrasound (HIFU), cryo, or radiation, Histotripsy relies on the mechanical action of cavitation for tissue destruction and not on heat, cold or ionizing energy.

### SUMMARY OF THE DISCLOSURE

A constraint device, comprising a patient section configured to be placed under a patient; and one or more peripheral sections extending from the patient section configured to support an expandable portion of an ultrasound coupling assembly and including one or more engagement features configured to attach the constraint device to a frame of the ultrasound coupling assembly.

In some aspects, the first and second peripheral sections include a mesh structure comprising a plurality of openings.

In one aspect, the constraint device comprises an open pore structure.

In some aspects, the first and second peripheral sections include a hexagonal pattern comprising a plurality of openings.

In some aspects, the plurality of openings are configured to engage with one or more attachment features of the rigid portion of the coupling interface.

In one aspect, the constraint device comprises an elastomeric material.

In other aspects, a flexibility of the constraint device is less than a flexibility of the expandable portion of the ultrasound coupling assembly.

In some aspects, at least the first section has a first mesh pattern, and the central section has a second mesh pattern.

In one aspect, the first mesh pattern is different than the second mesh pattern.

In some aspects, the first and second sections have a first width proximal to the central section and have a second, larger width distal to the central section.

In another aspect, the constraint device is generally bow-tie shaped.

An ultrasound coupling system is provided, comprising: a coupling container including one or more attachment features; a coupling membrane coupled to coupling container, the coupling container and coupling membrane forming leak-proof fluidic seal, the coupling membrane being configured to expand to conform to a patient when the coupling container and coupling membrane are filled with an ultrasound coupling medium; and a porous constraint device being configured to attach to the attachment features of the coupling container, the porous constraint device limiting expansion of the coupling membrane.

In some aspects, the porous constraint device is configured to limit expansion of the coupling membrane when the coupling membrane is filled with the ultrasound coupling medium.

In some aspects, the porous constraint device includes first and second sections having a mesh structure comprising a plurality of openings.

In another aspect, the porous constraint device comprises an open pore structure.

In some aspects, the first and second sections include a hexagonal pattern comprising a plurality of openings.

In other aspects, the porous constraint device comprises an elastomeric material.

In some aspects, an elasticity of the porous constraint device is less than an elasticity of the coupling membrane.

In another aspect, the first and second sections have a first mesh pattern, wherein the porous constraint device further comprises a central section between the first and second sections that has a second mesh pattern.

In some aspects, the first mesh pattern is different than the second mesh pattern.

In one aspect, the first and second sections have a first width proximal to the central section and have a second, larger width distal to the central section.

In other aspects, the constraint device is generally bow-tie shaped.

A method of mechanically supporting an ultrasound coupling assembly is provided, comprising: positioning the patient on a central section of a constraint device; positioning a rigid coupling container and coupling membrane over the patient; attaching first and second sides of the constraint device to the rigid coupling container; at least partially filling the rigid coupling container and coupling membrane with an ultrasound coupling medium; and preventing over-expansion of the coupling membrane with the constraint device.

A constraint system for use with a histotripsy system is provided, the constraint system comprising; an ultrasound coupling assembly comprising a rigid frame having attachment features disposed on an exterior surface therein, and an expandable membrane fixedly attached to the rigid frame; and a constraint device comprising a central section and one or more peripheral sections extending outward from the central section and including one or more engagement features configured to attach the constraint device to the attachment features.

### BRIEF DESCRIPTION OF THE DRAWINGS

The novel features of the invention are set forth with particularity in the claims that follow. A better understanding of the features and advantages of the present invention will be obtained by reference to the following detailed description that sets forth illustrative embodiments, in which the principles of the invention are utilized, and the accompanying drawings of which:

FIGS. 1A-1B illustrate an ultrasound imaging and therapy system.

FIG. 2 is one embodiment of a histotripsy therapy and imaging system with a coupling system.

FIGS. 3A-3E illustrate some embodiments of a membrane constraint.

FIGS. 3F-3G illustrate a close-up view of the embodiment of FIG. 3E.

FIG. 4 shows an example of a coupling assembly with attachment features for attachment to a membrane constraint.

FIG. 5 illustrates an example of a membrane constraint coupled to a coupling assembly.

FIGS. 6A-6B illustrate an embodiment of a coupling band of a coupling kit for removing air or bubbles from a coupling membrane.

FIGS. 7A-7B show a bubble removal tool of a coupling kit.

FIGS. 8A-8B show an inspection mirror of a coupling kit.

FIGS. 9A-9J show methods and techniques for packaging or folding the coupling kit.

FIG. 10 shows a coupling kit stored in a multi-tray retainer.

### DETAILED DESCRIPTION

The system, methods and devices of the disclosure may be used for open surgical, minimally invasive surgical (laparoscopic and percutaneous), robotic surgical (integrated into a robotically-enabled medical system), endoscopic or completely transdermal extracorporcal non-invasive acoustic cavitation for the treatment of healthy, diseased and/or injured tissue including but not limited to tissue destruction, cutting, skeletonizing and ablation. Furthermore, due to tissue selective properties, histotripsy may be used to create a cytoskeleton that allows for subsequent tissue regeneration either de novo or through the application of stem cells and other adjuvants. Finally, histotripsy can be used to cause the release of delivered agents such as chemotherapy and immunotherapy by locally causing the release of these agents by the application of acoustic energy to the targets. As will be described below, the acoustic cavitation system may include various sub-systems, including a Cart, Therapy, Integrated Imaging, Robotics, Coupling and Software. The system also may comprise various Other Components, Ancillaries and Accessories, including but not limited to computers, cables and connectors, networking devices, power supplies, displays, drawers/storage, doors, wheels, and various simulation and training tools, etc. All systems, methods and means creating/controlling/delivering histotripsy are considered to be a part of this disclosure, including new related inventions disclosed herein.

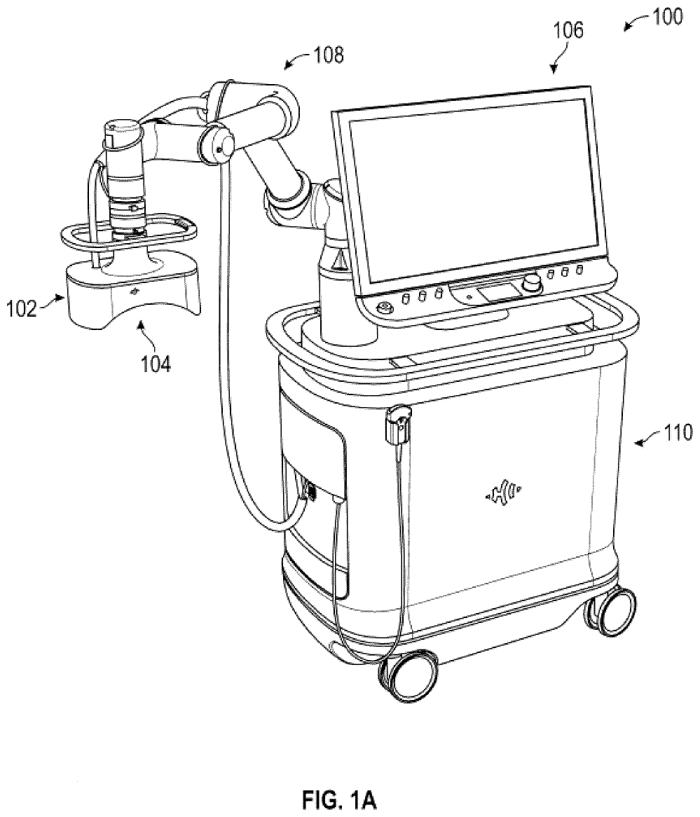

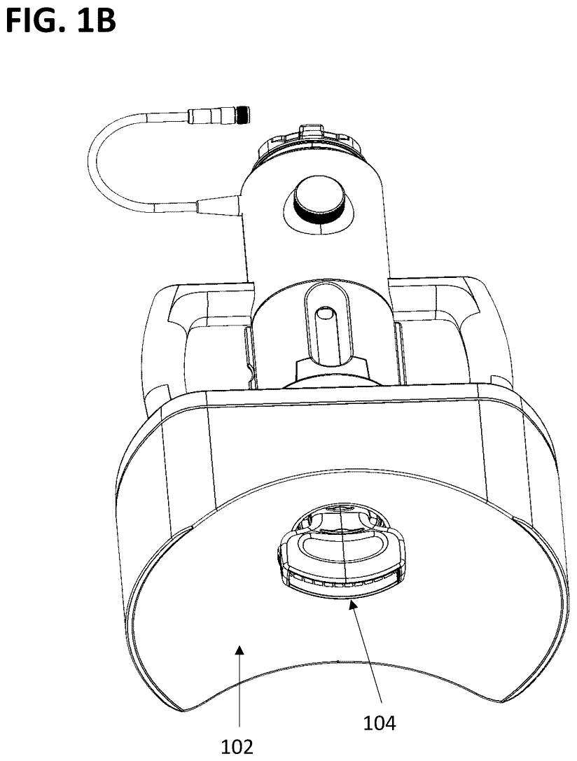

FIG. 1A generally illustrates histotripsy system 100 according to the present disclosure, comprising a therapy transducer 102, an imaging system 104, a display and control panel 106, a robotic positioning arm 108, and a cart 110. The system can further include an ultrasound coupling interface and a source of coupling medium, not shown.

FIG. 1B is a bottom view of the therapy transducer 102 and the imaging system 104. As shown, the imaging system can be positioned in the center of the therapy transducer. However, other embodiments can include the imaging system positioned in other locations within the therapy transducer, or even directly integrated into the therapy transducer. In some embodiments, the imaging system is configured to produce real-time imaging at a focal point of the therapy transducer. The system also allows for multiple imaging transducers to be located within the therapy transducer to provide multiple views of the target tissue simultaneously and to integrate these images into a single 3-D image.

The histotripsy system may comprise one or more of various sub-systems, including a Therapy sub-system that can create, apply, focus and deliver acoustic cavitation/histotripsy through one or more therapy transducers, Integrated Imaging sub-system (or connectivity to) allowing real-time visualization of the treatment site and histotripsy effect through-out the procedure, a Robotics positioning sub-system to mechanically and/or electronically steer the therapy transducer, further enabled to connect/support or interact with a Coupling sub-system to allow acoustic coupling between the therapy transducer and the patient, and Software to communicate, control and interface with the system and computer-based control systems (and other external systems) and various Other Components, Ancillaries and Accessories, including one or more user interfaces and displays, and related guided work-flows, all working in part or together. The system may further comprise various fluidics and fluid management components, including but not limited to, pumps, valve and flow controls, temperature and degassing controls, and irrigation and aspiration capabilities, as well as providing and storing fluids. It may also contain various power supplies and protectors.

As described above, the histotripsy system may include integrated imaging. However, in other embodiments, the histotripsy system can be configured to interface with separate imaging systems, such as C-arm, fluoroscope, cone beam CT, MRI, etc., to provide real-time imaging during histotripsy therapy. In some embodiments, the histotripsy system can be sized and configured to fit within a C-arm, fluoroscope, cone beam CT, MRI, etc.

### Cart

The Cart 110 of the histotripsy system may be generally configured in a variety of ways and form factors based on the specific uses and procedures. In some cases, systems may comprise multiple Carts, configured with similar or different arrangements. In some embodiments, the cart may be configured and arranged to be used in a radiology environment and in some cases in concert with imaging (e.g., CT, cone beam CT and/or MRI scanning). In other embodiments, it may be arranged for use in an operating room and a sterile environment for open surgical or laparoscopic surgical and endoscopic application, or in a robotically enabled operating room, and used alone, or as part of a surgical robotics procedure wherein a surgical robot conducts specific tasks before, during or after use of the system and delivery of acoustic cavitation/histotripsy. As such and depending on the procedure environment based on the aforementioned embodiments, the cart may be positioned to provide sufficient work-space and access to various anatomical locations on the patient (e.g., torso, abdomen, flank, head and neck, etc.), as well as providing work-space for other systems (e.g., anesthesia cart, laparoscopic tower, surgical robot, endoscope tower, etc.).

The Cart may also work with a patient surface (e.g., table or bed) to allow the patient to be presented and repositioned in a plethora of positions, angles and orientations, including allowing changes to such to be made pre, peri and post-procedurally. It may further comprise the ability to interface and communicate with one or more external imaging or image data management and communication systems, not limited to ultrasound, CT, fluoroscopy, cone beam CT, PET, PET/CT, MRI, optical, ultrasound, and image fusion and or image flow, of one or more modalities, to support the procedures and/or environments of use, including physical/mechanical interoperability (e.g., compatible within cone beam CT work-space for collecting imaging data pre, peri and/or post histotripsy) and to provide access to and display of patient medical data including but not limited to laboratory and historical medical record data.

In some embodiments one or more Carts may be configured to work together. As an example, one Cart may comprise a bedside mobile Cart equipped with one or more Robotic arms enabled with a Therapy transducer, and Therapy generator/amplifier, etc., while a companion cart working in concert and at a distance of the patient may comprise Integrated Imaging and a console/display for controlling the Robotic and Therapy facets, analogous to a surgical robot and master/slave configurations.

In some embodiments, the system may comprise a plurality of Carts, all slave to one master Cart, equipped to conduct acoustic cavitation procedures. In some arrangements and cases, one Cart configuration may allow for storage of specific sub-systems at a distance reducing operating room clutter, while another in concert Cart may comprise essentially bedside sub-systems and componentry (e.g., delivery system and therapy).

One can envision a plethora of permutations and configurations of Cart design, and these examples are in no way limiting the scope of the disclosure.

### Histotripsy

Histotripsy comprises short, high amplitude, focused ultrasound pulses to generate a dense, energetic, “bubble cloud”, capable of the targeted fractionation and destruction of tissue. Histotripsy is capable of creating controlled tissue erosion when directed at a tissue interface, including tissue/fluid interfaces, as well as well-demareated tissue fractionation and destruction, at sub-cellular levels, when it is targeted at bulk tissue. Unlike other forms of ablation, including thermal and radiation-based modalities, histotripsy does not rely on heat cold or ionizing (high) energy to treat tissue. Instead, histotripsy uses acoustic cavitation generated at the focus to mechanically effect tissue structure, and in some cases liquefy, suspend, solubilize and/or destruct tissue into sub-cellular components.

Histotripsy can be applied in various forms, including: 1) Intrinsic-Threshold Histotripsy: Delivers pulses with a 1-2 cycles of high amplitude negative/tensile phase pressure exceeding the intrinsic threshold to generate cavitation in the medium (e.g., ˜24-28 MPa for water-based soft tissue), 2) Shock-Scattering Histotripsy: Delivers typically pulses 3-20 cycles in duration. The shockwave (positive/compressive phase) scattered from an initial individual microbubble generated forms inverted shockwave, which constructively interfere with the incoming negative/tensile phase to form high amplitude negative/rarefactional phase exceeding the intrinsic threshold. In this way, a cluster of cavitation microbubbles is generated. The amplitude of the tensile phases of the pulses is sufficient to cause bubble nuclei in the medium to undergo inertial cavitation within the focal zone throughout the duration of the pulse. These nuclei scatter the incident shockwaves, which invert and constructively interfere with the incident wave to exceed the threshold for intrinsic nucleation, and 3) Boiling Histotripsy: Employs pulses roughly 1-20 ms in duration. Absorption of the shocked pulse rapidly heats the medium, thereby reducing the threshold for intrinsic nuclei. Once this intrinsic threshold coincides with the peak negative pressure of the incident wave, boiling bubbles form at the focus.

The large pressure generated at the focus causes a cloud of acoustic cavitation bubbles to form above certain thresholds, which creates localized stress and strain in the tissue and mechanical breakdown without significant heat deposition. At pressure levels where cavitation is not generated, minimal effect is observed on the tissue at the focus. This cavitation effect is observed only at pressure levels significantly greater than those which define the inertial cavitation threshold in water for similar pulse durations, on the order of 10 to 30 MPa peak negative pressure.

Histotripsy may be performed in multiple ways and under different parameters. It may be performed totally non-invasively by acoustically coupling a focused ultrasound transducer over the skin of a patient and transmitting acoustic pulses transcutaneously through overlying (and intervening) tissue to the focal zone (treatment zone and site). The application of histotripsy is not limited to a transdermal approach but can be applied through any means that allows contact of the transducer with tissue including open surgical laparoscopic surgical, percutaneous and robotically mediated surgical procedures. It may be further targeted, planned, directed and observed under direct visualization, via ultrasound imaging, given the bubble clouds generated by histotripsy may be visible as highly dynamic, echogenic regions on, for example, B Mode ultrasound images, allowing continuous visualization through its use (and related procedures). Likewise, the treated and fractionated tissue shows a dynamic change in echogenicity (typically a reduction), which can be used to evaluate, plan, observe and monitor treatment.

Generally, in histotripsy treatments, ultrasound pulses with 1 or more acoustic cycles are applied, and the bubble cloud formation relies on the pressure release scattering of the positive shock fronts (sometimes exceeding 100 MPa, P+) from initially initiated, sparsely distributed bubbles (or a single bubble). This is referred to as the “shock scattering mechanism”.

This mechanism depends on one (or a few sparsely distributed) bubble(s) initiated with the initial negative half cycle(s) of the pulse at the focus of the transducer. A cloud of microbubbles then forms due to the pressure release backscattering of the high peak positive shock fronts from these sparsely initiated bubbles. These back-scattered high-amplitude rarefactional waves exceed the intrinsic threshold thus producing a localized dense bubble cloud. Each of the following acoustic cycles then induces further cavitation by the backscattering from the bubble cloud surface, which grows or expands (the bubble cloud) towards the transducer. As a result, an elongated dense bubble cloud growing along the acoustic axis opposite the ultrasound propagation direction is observed with the shock scattering mechanism. This shock scattering process makes the bubble cloud generation not only dependent on the peak negative pressure, but also the number of acoustic cycles and the amplitudes of the positive shocks. Without at least one intense shock front developed by nonlinear propagation, no dense bubble clouds are generated when the peak negative half-cycles are below the intrinsic threshold.

When ultrasound pulses less than 2 cycles are applied, shock scattering can be minimized, and the generation of a dense bubble cloud depends on the negative half cycle(s) of the applied ultrasound pulses exceeding an “intrinsic threshold” of the medium. This is referred to as the “intrinsic threshold mechanism”.

This threshold can be in the range of 26-30 MPa for soft tissues with high water content, such as tissues in the human body. In some embodiments, using this intrinsic threshold mechanism, the spatial extent of the lesion may be well-defined and more predictable. With peak negative pressures (P−) not significantly higher than this threshold, sub-wavelength reproducible lesions as small as half of the −6 dB beam width of a transducer may be generated.

With high-frequency Histotripsy pulses, the size of the smallest reproducible lesion becomes smaller, which is beneficial in applications that require precise lesion generation. However, high-frequency pulses are more susceptible to attenuation and aberration, rendering problematical treatments at a larger penetration depth (e.g., ablation deep in the body) or through a highly aberrative medium (e.g., transcranial procedures, or procedures in which the pulses are transmitted through bone(s)). Histotripsy may further also be applied as a low-frequency “pump” pulse (typically <2 cycles and having a frequency between 100 kHz and 1 MHZ) can be applied together with a high-frequency “probe” pulse (typically <2 cycles and having a frequency greater than 2 MHZ, or ranging between 2 MHz and 10 MHZ) wherein the peak negative pressures of the low and high-frequency pulses constructively interfere to exceed the intrinsic threshold in the target tissue or medium. The low-frequency pulse, which is more resistant to attenuation and aberration, can raise the peak negative pressure P− level for a region of interest (ROI), while the high-frequency pulse, which provides more precision, can pin-point a targeted location within the ROI and raise the peak negative pressure P− above the intrinsic threshold. This approach may be referred to as “dual frequency”, “dual beam histotripsy” or “parametric histotripsy.”

Additional systems, methods and parameters to deliver optimized histotripsy, using shock scattering, intrinsic threshold, and various parameters enabling frequency compounding and bubble manipulation, are herein included as part of the system and methods disclosed herein, including additional means of controlling said histotripsy effect as pertains to steering and positioning the focus, and concurrently managing tissue effects (e.g., prefocal thermal collateral damage) at the treatment site or within intervening tissue. Further, it is disclosed that the various systems and methods, which may include a plurality of parameters, such as but not limited to, frequency, operating frequency, center frequency, pulse repetition frequency, pulses, bursts, number of pulses, cycles, length of pulses, amplitude of pulses, pulse period, delays, burst repetition frequency, sets of the former, loops of multiple sets, loops of multiple and/or different sets, sets of loops, and various combinations or permutations of, etc., are included as a part of this disclosure, including future envisioned embodiments of such.

### Therapy Components

The Therapy sub-system may work with other sub-systems to create, optimize, deliver, visualize, monitor and control acoustic cavitation, also referred to herein and in following as “histotripsy”, and its derivatives of, including boiling histotripsy and other thermal high frequency ultrasound approaches. It is noted that the disclosed inventions may also further benefit other acoustic therapies that do not comprise a cavitation, mechanical or histotripsy component. The therapy sub-system can include, among other features, an ultrasound therapy transducer and a pulse generator system configured to deliver ultrasound pulses into tissue.

In order to create and deliver histotripsy and derivatives of histotripsy, the therapy sub-system may also comprise components, including but not limited to, one or more function generators, amplifiers, therapy transducers and power supplies.

The therapy transducer can comprise a single element or multiple elements configured to be excited with high amplitude electric pulses (>1000V or any other voltage that can cause harm to living organisms). The amplitude necessary to drive the therapy transducers for Histotripsy vary depending on the design of the transducer and the materials used (e.g., solid or polymer/piezoelectric composite including ceramic or single crystal) and the transducer center frequency which is directly proportional to the thickness of the piezo-electric material. Transducers therefore operating at a high frequency require lower voltage to produce a given surface pressure than is required by low frequency therapy transducers. In some embodiments, the transducer elements are formed using a piezoelectric-polymer composite material or a solid piezoelectric material. Further, the piezoelectric material can be of polycrystalline/ceramic or single crystalline formulation. In some embodiments the transducer elements can be formed using silicon using MEMs technology, including CMUT and PMUT designs.

In some embodiments, the function generator may comprise a field programmable gate array (FPGA) or other suitable function generator. The FPGA may be configured with parameters disclosed previously herein, including but not limited to frequency, pulse repetition frequency, bursts, burst numbers, where bursts may comprise pulses, numbers of pulses, length of pulses, pulse period, delays, burst repetition frequency or period, where sets of bursts may comprise a parameter set, where loop sets may comprise various parameter sets, with or without delays, or varied delays, where multiple loop sets may be repeated and/or new loop sets introduced, of varied time delay and independently controlled, and of various combinations and permutations of such, overall and throughout.

In some embodiments, the generator or amplifier may be configured to be a universal single-cycle or multi-cycle pulse generator, and to support driving via Class D or inductive driving, as well as across all envisioned clinical applications, use environments, also discussed in part later in this disclosure. In other embodiments, the class D or inductive current driver may be configured to comprise transformer and/or auto-transformer driving circuits to further provide step up/down components, and in some cases, to preferably allow a step up in the amplitude. They may also comprise specific protective features, to further support the system, and provide capability to protect other parts of the system (e.g., therapy transducer and/or amplifier circuit components) and/or the user, from various hazards, including but not limited to, electrical safety hazards, which may potentially lead to use environment, system and therapy system, and user harms, damage or issues.

Disclosed generators may allow and support the ability of the system to select, vary and control various parameters (through enabled software tools), including, but not limited to those previously disclosed, as well as the ability to start/stop therapy, set and read voltage level, pulse and/or burst repetition frequency, number of cycles, duty ratio, channel enabled and delay, etc., modulate pulse amplitude on a fast time-scale independent of a high voltage supply, and/or other service, diagnostic or treatment features.

In some embodiments, the Therapy sub-system and/or components of, such as the amplifier, may comprise further integrated computer processing capability and may be networked, connected, accessed, and/or be removable/portable, modular, and/or exchangeable between systems, and/or driven/commanded from/by other systems, or in various combinations. Other systems may include other acoustic cavitation/histotripsy, HIFU, HITU, radiation therapy, radiofrequency, microwave, and cryoablation systems, navigation and localization systems, open surgical, laparoscopic, single incision/single port, endoscopic and non-invasive surgical robots, laparoscopic or surgical towers comprising other energy-based or vision systems, surgical system racks or booms, imaging carts, etc.

In some embodiments, one or more amplifiers may comprise a Class D amplifier and related drive circuitry including matching network components. Depending on the transducer element electric impedance and choice of the matching network components (e.g., an LC circuit made of an inductor LI in series and the capacitor C1 in parallel), the combined impedance can be aggressively set low in order to have high amplitude electric waveform necessary to drive the transducer element. The maximum amplitude that Class D amplifiers is dependent on the circuit components used, including the driving MOSFET/IGBT transistors, matching network components or inductor, and transformer or autotransformer, and of which may be typically in the low kV (e.g., 1-3 kV) range.

Therapy transducer element(s) are excited with an electrical waveform with an amplitude (voltage) to produce a pressure output sufficient for Histotripsy therapy. The excitation electric field can be defined as the necessary waveform voltage per thickness of the piezoelectric element. For example, because a piezoelectric element operating at 1 MHZ transducer is half the thickness of an equivalent 500 kHz element, it will require half the voltage to achieve the same electric field and surface pressure.

The Therapy sub-system may also comprise therapy transducers of various designs and working parameters, supporting use in various procedures (and procedure settings). Systems may be configured with one or more therapy transducers, that may be further interchangeable, and work with various aspects of the system in similar or different ways (e.g., may interface to a robotic arm using a common interface and exchange feature, or conversely, may adapt to work differently with application specific imaging probes, where different imaging probes may interface and integrate with a therapy transducer in specifically different ways).

Therapy transducers may be configured of various parameters that may include size, shape (e.g., rectangular or round; anatomically curved housings, etc.), geometry, focal length, number of elements, size of elements, distribution of elements (e.g., number of rings, size of rings for annular patterned transducers), frequency, enabling electronic beam steering, etc. Transducers may be composed of various materials (e.g., piezoelectric, silicon, etc.), form factors and types (e.g., machined elements, chip-based, etc.) and/or by various methods of fabrication of.

Transducers may be designed and optimized for clinical applications (e.g., abdominal tumors, peripheral vascular disease, fat ablation, etc.) and desired outcomes (e.g., acoustic cavitation/histotripsy without thermal injury to intervening tissue), and affording a breadth of working ranges, including relatively shallow and superficial targets (e.g., thyroid or breast nodules), versus, deeper or harder to reach targets, such as central liver or brain tumors. They may be configured to enable acoustic cavitation/histotripsy under various parameters and sets of, as enabled by the aforementioned system components (e.g., function generator and amplifier, etc.), including but not limited to frequency, pulse repetition rate, pulses, number of pulses, pulse length, pulse period, delays, repetitions, sync delays, sync period, sync pulses, sync pulse delays, various loop sets, others, and permutations of. The transducer may also be designed to allow for the activation of a drug payload either deposited in tissue through various means including injection, placement or delivery in micelle or nanostructures.

### Integrated Imaging

The disclosed system may comprise various imaging modalities to allow users to visualize, monitor and collect/use feedback of the patient's anatomy, related regions of interest and treatment/procedure sites, as well as surrounding and intervening tissues to assess, plan and conduct procedures, and adjust treatment parameters as needed. Imaging modalities may comprise various ultrasound, x-ray, CT, MRI, PET, fluoroscopy, optical, contrast or agent enhanced versions, and/or various combinations of. It is further disclosed that various image processing and characterization technologies may also be utilized to afford enhanced visualization and user decision making. These may be selected or commanded manually by the user or in an automated fashion by the system. The system may be configured to allow side by side, toggling, overlays, 3D reconstruction, segmentation, registration, multi-modal image fusion, image flow, and/or any methodology affording the user to identify, define and inform various aspects of using imaging during the procedure, as displayed in the various system user interfaces and displays. Examples may include locating, displaying and characterizing regions of interest, organ systems, potential treatment sites within, with on and/or surrounding organs or tissues, identifying critical structures such as ducts, vessels, nerves, ureters, fissures, capsules, tumors, tissue trauma/injury/disease, other organs, connective tissues, etc., and/or in context to one another, of one or more (e.g., tumor draining lymphatics or vasculature; or tumor proximity to organ capsule or underlying other organ), as unlimited examples.

Systems may be configured to include onboard integrated imaging hardware, software, sensors, probes and wetware, and/or may be configured to communicate and interface with external imaging and image processing systems. The aforementioned components may be also integrated into the system's Therapy sub-system components wherein probes, imaging arrays, or the like, and electrically, mechanically or electromechanically integrated into therapy transducers. This may afford, in part, the ability to have geometrically aligned imaging and therapy, with the therapy directly within the field of view, and in some cases in line, with imaging. In some embodiments, this integration may comprise a fixed orientation of the imaging capability (e.g., imaging probe) in context to the therapy transducer. In other embodiments, the imaging solution may be able to move or adjust its position, including modifying angle, extension (e.g., distance from therapy transducer or patient), rotation (e.g., imaging plane in example of an ultrasound probe) and/or other parameters, including moving/adjusting dynamically while actively imaging. The imaging component or probe may be encoded so its orientation and position relative to another aspect of the system, such as the therapy transducer, and/or robotically-enabled positioning component may be determined.

In one embodiment, the system may comprise onboard ultrasound, further configured to allow users to visualize, monitor and receive feedback for procedure sites through the system displays and software, including allowing ultrasound imaging and characterization (and various forms of), ultrasound guided planning and ultrasound guided treatment, all in real-time. The system may be configured to allow users to manually, semi-automated or in fully automated means image the patient (e.g., by hand or using a robotically-enabled imager).

In some embodiments, imaging feedback and monitoring can include monitoring changes in: backscatter from bubble clouds; speckle reduction in backscatter; backscatter speckle statistics; mechanical properties of tissue (i.e., elastography); tissue perfusion (i.e., ultrasound contrast); shear wave propagation; acoustic emissions, electrical impedance tomography, and/or various combinations of, including as displayed or integrated with other forms of imaging (e.g., CT or MRI).

In some embodiments, imaging including feedback and monitoring from backscatter from bubble clouds, may be used as a method to determine immediately if the histotripsy process has been initiated, is being properly maintained, or even if it has been extinguished. For example, this method enables continuously monitored in real time drug delivery, tissue erosion, and the like. The method also can provide feedback permitting the histotripsy process to be initiated at a higher intensity and maintained at a much lower intensity. For example, backscatter feedback can be monitored by any transducer or ultrasonic imager. By measuring feedback for the therapy transducer, an accessory transducer can send out interrogation pulses or be configured to passively detect cavitation. Moreover, the nature of the feedback received can be used to adjust acoustic parameters (and associated system parameters) to optimize the drug delivery and/or tissue erosion process.

In some embodiments, imaging including feedback and monitoring from backscatter, and speckle reduction, may be configured in the system.

For systems comprising feedback and monitoring via backscattering, and as means of background, as tissue is progressively mechanically subdivided, in other words homogenized, disrupted, or eroded tissue, this process results in changes in the size and distribution of acoustic scatter. At some point in the process, the scattering particle size and density is reduced to levels where little ultrasound is scattered, or the amount scattered is reduced significantly. This results in a significant reduction in speckle, which is the coherent constructive and destructive interference patterns of light and dark spots seen on images when coherent sources of illumination are used; in this case, ultrasound. After some treatment time, the speckle reduction results in a dark area in the therapy volume. Since the amount of speckle reduction is related to the amount of tissue subdivision, it can be related to the size of the remaining tissue fragments. When this size is reduced to sub-cellular levels, no cells are assumed to have survived. So, treatment can proceed until a desired speckle reduction level has been reached. Speckle is easily seen and evaluated on standard ultrasound imaging systems. Specialized transducers and systems, including those disclosed herein, may also be used to evaluate the backscatter changes.

Further, systems comprising feedback and monitoring via speckle, and as means of background, an image may persist from frame to frame and change very little as long as the scatter distribution does not change and there is no movement of the imaged object. However, long before the scatters are reduced enough in size to cause speckle reduction, they may be changed sufficiently to be detected by signal processing and other means. This family of techniques can operate as detectors of speckle statistics changes. For example, the size and position of one or more speckles in an image will begin to decorrelate before observable speckle reduction occurs. Speckle decorrelation, after appropriate motion compensation, can be a sensitive measure of the mechanical disruption of the tissues, and thus a measure of therapeutic efficacy. This feedback and monitoring technique may permit early observation of changes resulting from the acoustic cavitation/histotripsy process and can identify changes in tissue before substantial or complete tissue effect (e.g., erosion occurs). In one embodiment, this method may be used to monitor the acoustic cavitation/histotripsy process for enhanced drug delivery where treatment sites/tissue is temporally disrupted, and tissue damage/erosion is not desired. In other embodiments, this may comprise speckle decorrelation by movement of scatters in an increasingly fluidized therapy volume. For example, in the case where partial or complete tissue erosion is desired.

For systems comprising feedback and monitoring via elastography, and as means of background, as treatment sites/tissue are further subdivided per an acoustic cavitation/histotripsy effect (homogenized, disrupted, or eroded), its mechanical properties change from a soft but interconnected solid to a viscous fluid or paste with few long-range interactions. These changes in mechanical properties can be measured by various imaging modalities including MRI and ultrasound imaging systems. For example, an ultrasound pulse can be used to produce a force (i.e., a radiation force) on a localized volume of tissue. The tissue response (displacements, strains, and velocities) can change significantly during histotripsy treatment allowing the state of tissue disruption to be determined by imaging or other quantitative means.

Systems may also comprise feedback and monitoring via shear wave propagation changes. As means of background, the subdivision of tissues makes the tissue more fluid and less solid and fluid systems generally do not propagate shear waves. Thus, the extent of tissue fluidization provides opportunities for feedback and monitoring of the histotripsy process. For example, ultrasound and MRI imaging systems can be used to observe the propagation of shear waves. The extinction of such waves in a treated volume is used as a measure of tissue destruction or disruption. In one system embodiment, the system and supporting sub-systems may be used to generate and measure the interacting shear waves. For example, two adjacent ultrasound foci might perturb tissue by pushing it in certain ways. If adjacent foci are in a fluid, no shear waves propagate to interact with each other. If the tissue is not fluidized, the interaction would be detected with external means, for example, by a difference frequency only detected when two shear waves interact nonlinearly, with their disappearance correlated to tissue damage. As such, the system may be configured to use this modality to enhance feedback and monitoring of the acoustic cavitation/histotripsy procedure.

For systems comprising feedback and monitoring via acoustic emission, and as means of background, as a tissue volume is subdivided, its effect on acoustic cavitation/histotripsy (e.g., the bubble cloud here) is changed. For example, bubbles may grow larger and have a different lifetime and collapse changing characteristics in intact versus fluidized tissue. Bubbles may also move and interact after tissue is subdivided producing larger bubbles or cooperative interaction among bubbles, all of which can result in changes in acoustic emission. These emissions can be heard during treatment and they change during treatment. Analysis of these changes, and their correlation to therapeutic efficacy, enables monitoring of the progress of therapy, and may be configured as a feature of the system.

For systems comprising feedback and monitoring via electrical impedance tomography, and as means of background, an impedance map of a therapy site can be produced based upon the spatial electrical characteristics throughout the therapy site. Imaging of the conductivity or permittivity of the therapy site of a patient can be inferred from taking skin surface electrical measurements. Conducting electrodes are attached to a patient's skin and small alternating currents are applied to some or all of the electrodes. One or more known currents are injected into the surface and the voltage is measured at a number of points using the electrodes. The process can be repeated for different configurations of applied current. The resolution of the resultant image can be adjusted by changing the number of electrodes employed. A measure of the electrical properties of the therapy site within the skin surface can be obtained from the impedance map, and changes in and location of the acoustic cavitation/histotripsy (e.g., bubble cloud, specifically) and histotripsy process can be monitored using this as configured in the system and supporting sub-systems.

The user may be allowed to further select, annotate, mark, highlight, and/or contour, various regions of interest or treatment sites, and defined treatment targets (on the image(s)), of which may be used to command and direct the system where to image, test and/or treat, through the system software and user interfaces and displays. In some arrangements, the user may use a manual ultrasound probe (e.g., diagnostic hand-held probe) to conduct the procedure. In another arrangement, the system may use a robot and/or electromechanical positioning system to conduct the procedure, as directed and/or automated by the system, or conversely, the system can enable combinations of manual and automated uses.

The system may further include the ability to conduct image registration, including imaging and image data set registration to allow navigation and localization of the system to the patient, including the treatment site (e.g., tumor, critical structure, bony anatomy, anatomy and identifying features of, etc.). In one embodiment, the system allows the user to image and identify a region of interest, for example the liver, using integrated ultrasound, and to select and mark a tumor (or surrogate marker of) comprised within the liver through/displayed in the system software, and wherein said system registers the image data to a coordinate system defined by the system, that further allows the system's Therapy and Robotics sub-systems to deliver synchronized acoustic cavitation/histotripsy to said marked tumor. The system may comprise the ability to register various image sets, including those previously disclosed, to one another, as well as to afford navigation and localization (e.g., of a therapy transducer to a CT or MRI/ultrasound fusion image with the therapy transducer and Robotics sub-system tracking to said image).

The system may also comprise the ability to work in a variety of interventional, endoscopic and surgical environments, including alone and with other systems (surgical/laparoscopic towers, vision systems, endoscope systems and towers, ultrasound enabled endoscopic ultrasound (flexible and rigid), percutaneous/endoscopic/laparoscopic and minimally invasive navigation systems (e.g., optical, electromagnetic, shape-sensing, ultrasound-enabled, etc.), of also which may work with, or comprise various optical imaging capabilities (e.g., fiber and or digital). The disclosed system may be configured to work with these systems, in some embodiments working alongside them in concert, or in other embodiments where all or some of the system may be integrated into the above systems/platforms (e.g., acoustic cavitation/histotripsy-enabled endoscope system or laparoscopic surgical robot). In many of these environments, a therapy transducer may be utilized at or around the time of use, for example, of an optically guided endoscope/bronchoscope, or as another example, at the time a laparoscopic robot (e.g., Intuitive Da Vinci* Xi system) is viewing/manipulating a tissue/treatment site. Further, these embodiments and examples may include where said other systems/platforms are used to deliver (locally) fluid to enable the creation of a man-made acoustic window, where on under normal circumstances may not exist (e.g., fluidizing a segment or lobe of the lung in preparation for acoustic cavitation/histotripsy via non-invasive transthoracic treatment (e.g., transducer externally placed on/around patient). Systems disclosed herein may also comprise all or some of their sub-system hardware packaged within the other system cart/console/systems described here (e.g., acoustic cavitation/histotripsy system and/or sub-systems integrated and operated from said navigation or laparoscopic system).

The system may also be configured, through various aforementioned parameters and other parameters, to display real-time visualization of a bubble cloud in a spatial-temporal manner, including the resulting tissue effect peri/post-treatment from tissue/bubble cloud interaction, wherein the system can dynamically image and visualize, and display, the bubble cloud, and any changes to it (e.g., decreasing or increasing echogenicity), which may include intensity, shape, size, location, morphology, persistence, etc. These features may allow users to continuously track and follow the treatment in real-time in one integrated procedure and interface/system, and confirm treatment safety and efficacy on the fly (versus other interventional or surgical modalities, which either require multiple procedures to achieve the same, or where the treatment effect is not visible in real-time (e.g., radiation therapy), or where it is not possible to achieve such (e.g., real-time visualization of local tissue during thermal ablation), and/or where the other procedure further require invasive approaches (e.g., incisions or punctures) and iterative imaging in a scanner between procedure steps (e.g., CT or MRI scanning). The above disclosed systems, sub-systems, components, modalities, features and work-flows/methods of use may be implemented in an unlimited fashion through enabling hardware, software, user interfaces and use environments, and future improvements, enhancements and inventions in this area are considered as included in the scope of this disclosure, as well as any of the resulting data and means of using said data for analytics, artificial intelligence or digital health applications and systems.

### Robotics

They system may comprise various Robotic sub-systems and components, including but not limited to, one or more robotic arms and controllers, which may further work with other sub-systems or components of the system to deliver and monitor acoustic cavitation/histotripsy. As previously discussed herein, robotic arms and control systems may be integrated into one or more Cart configurations.

For example, one system embodiment may comprise a Cart with an integrated robotic arm and control system, and Therapy, Integrated Imaging and Software, where the robotic arm and other listed sub-systems are controlled by the user through the form factor of a single bedside Cart.

In other embodiments, the Robotic sub-system may be configured in one or more separate Carts, that may be a driven in a master/slave configuration from a separate master or Cart, wherein the robotically-enabled Cart is positioned bed/patient-side, and the Master is at a distance from said Cart.

Disclosed robotic arms may be comprised of a plurality of joints, segments, and degrees of freedom and may also include various integrated sensor types and encoders, implemented for various use and safety features. Sensing technologies and data may comprise, as an example, vision, potentiometers, position/localization, kinematics, force, torque, speed, acceleration, dynamic loading, and/or others. In some cases, sensors may be used for users to direct robot commands (e.g., hand gesture the robot into a preferred set up position, or to dock home). Additional details on robotic arms can be found in US Patent Pub. No. 2013/0255426 to Kassow et al. which is disclosed herein by reference in its entirety.

The robotic arm receives control signals and commands from the robotic control system, which may be housed in a Cart. The system may be configured to provide various functionalities, including but not limited to, position, tracking, patterns, triggering, and events/actions.

Position may be configured to comprise fixed positions, pallet positions, time-controlled positions, distance-controlled positions, variable-time controlled positions, variable-distance controlled positions.

Tracking may be configured to comprise time-controlled tracking and/or distance-controlled tracking.

The patterns of movement may be configured to comprise intermediate positions or waypoints, as well as sequence of positions, through a defined path in space.

Triggers may be configured to comprise distance measuring means, time, and/or various sensor means including those disclosed herein, and not limited to, visual/imaging-based, force, torque, localization, energy/power feedback and/or others.

Events/actions may be configured to comprise various examples, including proximity-based (approaching/departing a target object), activation or de-activation of various end-effectors (e.g., therapy transducers), starting/stopping/pausing sequences of said events, triggering or switching between triggers of events/actions, initiating patterns of movement and changing/toggling between patterns of movement, and/or time-based and temporal over the defined work and time-space.

In one embodiment, the system comprises a three degree of freedom robotic positioning system, enabled to allow the user (through the software of the system and related user interfaces), to micro-position a therapy transducer through X, Y, and Z coordinate system, and where gross macro-positioning of the transducer (e.g., aligning the transducer on the patient's body) is completed manually. In some embodiments, the robot may comprise 6 degrees of freedom including X, Y, Z, and pitch, roll and yaw. In other embodiments, the Robotic sub-system may comprise further degrees of freedom, that allow the robot arm supporting base to be positioned along a linear axis running parallel to the general direction of the patient surface, and/or the supporting base height to be adjusted up or down, allowing the position of the robotic arm to be modified relative to the patient, patient surface, Cart, Coupling sub-system, additional robots/robotic arms and/or additional surgical systems, including but not limited to, surgical towers, imaging systems, endoscopic/laparoscopic systems, and/or other.

One or more robotic arms may also comprise various features to assist in maneuvering and modifying the arm position, manually or semi-manually, and of which said features may interface on or between the therapy transducer and the most distal joint of the robotic arm. In some embodiments, the feature is configured to comprise a handle allowing maneuvering and manual control with one or more hands. The handle may also be configured to include user input and electronic control features of the robotic arm, to command various drive capabilities or modes, to actuate the robot to assist in gross or fine positioning of the arm (e.g., activating or deactivating free drive mode). The work-flow for the initial positioning of the robotic arm and therapy head can be configured to allow either first positioning the therapy transducer/head in the coupling solution, with the therapy transducer directly interfaced to the arm, or in a different work-flow, allowing the user to set up the coupling solution first, and enabling the robot arm to be interfaced to the therapy transducer/coupling solution as a later/terminal set up step.

In some embodiments, the robotic arm may comprise a robotic arm on a laparoscopic, single port, endoscopic, hybrid or combination of, and/or other robot, wherein said robot of the system may be a slave to a master that controls said arm, as well as potentially a plurality of other arms, equipped to concurrently execute other tasks (vision, imaging, grasping, cutting, ligating, sealing, closing, stapling, ablating, suturing, marking, etc.), including actuating one or more laparoscopic arms (and instruments) and various histotripsy system components. For example, a laparoscopic robot may be utilized to prepare the surgical site, including manipulating organ position to provide more ideal acoustic access and further stabilizing said organ in some cases to minimize respiratory motion. In conjunction and parallel to this, a second robotic arm may be used to deliver non-invasive acoustic cavitation through a body cavity, as observed under real-time imaging from the therapy transducer (e.g., ultrasound) and with concurrent visualization via a laparoscopic camera. In other related aspects, a similar approach may be utilized with a combination of an endoscopic and non-invasive approach, and further, with a combination of an endoscopic, laparoscopic and non-invasive approach.

### Software

The system may comprise various software applications, features and components which allow the user to interact, control and use the system for a plethora of clinical applications. The Software may communicate and work with one or more of the sub-systems, including but not limited to Therapy, Integrated Imaging, Robotics and Other Components, Ancillaries and Accessories of the system.

Overall, in no specific order of importance, the software may provide features and support to initialize and set up the system, service the system, communicate and import/export/store data, modify/manipulate/configure/control/command various settings and parameters by the user, mitigate safety and use-related risks, plan procedures, provide support to various configurations of transducers, robotic arms and drive systems, function generators and amplifier circuits/slaves, test and treatment ultrasound sequences, transducer steering and positioning (electromechanical and electronic beam steering, etc.), treatment patterns, support for imaging and imaging probes, manual and electromechanical/robotically-enabling movement of, imaging support for measuring/characterizing various dimensions within or around procedure and treatment sites (e.g., depth from one anatomical location to another, etc., pre-treatment assessments and protocols for measuring/characterizing in situ treatment site properties and conditions (e.g., acoustic cavitation/histotripsy thresholds and heterogeneity of), targeting and target alignment, calibration, marking/annotating, localizing/navigating, registering, guiding, providing and guiding through work-flows, procedure steps, executing treatment plans and protocols autonomously, autonomously and while under direct observation and viewing with real-time imaging as displayed through the software, including various views and viewports for viewing, communication tools (video, audio, sharing, etc.), troubleshooting, providing directions, warnings, alerts, and/or allowing communication through various networking devices and protocols. It is further envisioned that the software user interfaces and supporting displays may comprise various buttons, commands, icons, graphics, text, etc., that allow the user to interact with the system in a user-friendly and effective manner, and these may be presented in an unlimited number of permutations, layouts and designs, and displayed in similar or different manners or feature sets for systems that may comprise more than one display (e.g., touch screen monitor and touch pad), and/or may network to one or more external displays or systems (e.g., another robot, navigation system, system tower, console, monitor, touch display, mobile device, tablet, etc.).

The software, as a part of a representative system, including one or more computer processors, may support the various aforementioned function generators (e.g., FPGA), amplifiers, power supplies and therapy transducers. The software may be configured to allow users to select, determine and monitor various parameters and settings for acoustic cavitation/histotripsy, and upon observing/receiving feedback on performance and conditions, may allow the user to stop/start/modify said parameters and settings.

The software may be configured to allow users to select from a list or menu of multiple transducers and support the auto-detection of said transducers upon connection to the system (and verification of the appropriate sequence and parameter settings based on selected application). In other embodiments, the software may update the targeting and amplifier settings (e.g., channels) based on the specific transducer selection. The software may also provide transducer recommendations based on pre-treatment and planning inputs. Conversely, the software may provide error messages or warnings to the user if said therapy transducer, amplifier and/or function generator selections or parameters are erroneous, yield a fault or failure. This may further comprise reporting the details and location of such.

In addition to above, the software may be configured to allow users to select treatment sequences and protocols from a list or menu, and to store selected and/or previous selected sequences and protocols as associated with specific clinical uses or patient profiles. Related profiles may comprise any associated patient, procedure, clinical and/or engineering data, and maybe used to inform, modify and/or guide current or future treatments or procedures/interventions, whether as decision support or an active part of a procedure itself (e.g., using serial data sets to build and guide new treatments).

As a part of planning or during the treatment, the software (and in working with other components of the system) may allow the user to evaluate and test acoustic cavitation/histotripsy thresholds at various locations in a user-selected region of interest or defined treatment area/volume, to determine the minimum cavitation thresholds throughout said region or area/volume, to ensure treatment parameters are optimized to achieve, maintain and dynamically control acoustic cavitation/histotripsy. In one embodiment, the system allows a user to manually evaluate and test threshold parameters at various points. Said points may include those at defined boundary, interior to the boundary and center locations/positions, of the selected region of interest and treatment area/volume, and where resulting threshold measurements may be reported/displayed to the user, as well as utilized to update therapy parameters before treatment. In another embodiment, the system may be configured to allow automated threshold measurements and updates, as enabled by the aforementioned Robotics sub-system, wherein the user may direct the robot, or the robot may be commanded to execute the measurements autonomously.

Software may also be configured, by working with computer processors and one or more function generators, amplifiers and therapy transducers, to allow various permutations of delivering and positioning optimized acoustic cavitation/histotripsy in and through a selected area/volume. This may include, but not limited to, systems configured with a fixed/natural focus arrangement using purely electromechanical positioning configuration(s), electronic beam steering (with or without electromechanical positioning), electronic beam steering to a new selected fixed focus with further electromechanical positioning, axial (Z axis) electronic beam steering with lateral (X and Y) electromechanical positioning, high speed axial electronic beam steering with lateral electromechanical positioning, high speed beam steering in 3D space, various combinations of including with dynamically varying one or more acoustic cavitation/histotripsy parameters based on the aforementioned ability to update treatment parameters based on threshold measurements (e.g., dynamically adjusting amplitude across the treatment area/volume).

### Other Components, Ancillaries and Accessories

The system may comprise various other components, ancillaries and accessories, including but not limited to computers, computer processors, power supplies including high voltage power supplies, controllers, cables, connectors, networking devices, software applications for security, communication, integration into information systems including hospital information systems, cellular communication devices and modems, handheld wired or wireless controllers, goggles or glasses for advanced visualization, augmented or virtual reality applications, cameras, sensors, tablets, smart devices, phones, internet of things enabling capabilities, specialized use “apps” or user training materials and applications (software or paper based), virtual proctors or trainers and/or other enabling features, devices, systems or applications, and/or methods of using the above.

### System Variations and Methods/Applications

In addition to performing a breadth of procedures, the system may allow additional benefits, such as enhanced planning, imaging and guidance to assist the user. In one embodiment, the system may allow a user to create a patient, target and application specific treatment plan, wherein the system may be configured to optimize treatment parameters based on feedback to the system during planning, and where planning may further comprise the ability to run various test protocols to gather specific inputs to the system and plan.

Feedback may include various energy, power, location, position, tissue and/or other parameters.

The system, and the above feedback, may also be further configured and used to autonomously (and robotically) execute the delivery of the optimized treatment plan and protocol, as visualized under real-time imaging during the procedure, allowing the user to directly observe the local treatment tissue effect, as it progresses through treatment, and start/stop/modify treatment at their discretion. Both test and treatment protocols may be updated over the course of the procedure at the direction of the user, or in some embodiments, based on logic embedded within the system.

It is also recognized that many of these benefits may further improve other forms of acoustic therapy, including thermal ablation with high intensity focused ultrasound (HIFU), high intensity therapeutic ultrasound (HITU) including boiling histotripsy (thermal cavitation), and are considered as part of this disclosure. The disclosure also considers the application of histotripsy as a means to activate previously delivered in active drug payloads whose activity is inert due to protection in a micelle, nanostructure or similar protective structure or through molecular arrangement that allows activation only when struck with acoustic energy.

In another aspect, the Therapy sub-system, comprising in part, one or more amplifiers, transducers and power supplies, may be configured to allow multiple acoustic cavitation and histotripsy driving capabilities, affording specific benefits based on application, method and/or patient specific use. These benefits may include, but are not limited to, the ability to better optimize and control treatment parameters, which may allow delivery of more energy, with more desirable thermal profiles, increased treatment speed and reduced procedure times, enable electronic beam steering and/or other features.

This disclosure also includes novel systems and concepts as related to systems and sub-systems comprising new and “universal” amplifiers, which may allow multiple driving approaches (e.g., single and multi-cycle pulsing). In some embodiments, this may include various novel features to further protect the system and user, in terms of electrical safety or other hazards (e.g., damage to transducer and/or amplifier circuitry).

In another aspect, the system, and Therapy sub-system, may include a plethora of therapy transducers, where said therapy transducers are configured for specific applications and uses and may accommodate treating over a wide range of working parameters (target size, depth, location, etc.) and may comprise a wide range of working specifications (detailed below). Transducers may further adapt, interface and connect to a robotically-enabled system, as well as the Coupling sub-system, allowing the transducer to be positioned within, or along with, an acoustic coupling device allowing, in many embodiments, concurrent imaging and histotripsy treatments through an acceptable acoustic window. The therapy transducer may also comprise an integrated imaging probe or localization sensors, capable of displaying and determining transducer position within the treatment site and affording a direct field of view (or representation of) the treatment site, and as the acoustic cavitation/histotripsy tissue effect and bubble cloud may or may not change in appearance and intensity, throughout the treatment, and as a function of its location within said treatment (e.g., tumor, healthy tissue surrounding, critical structures, adipose tissue, etc.).

The systems, methods and use of the system disclosed herein, may be beneficial to overcoming significant unmet needs in the areas of soft tissue ablation, oncology, immuno-oncology, advanced image guided procedures, surgical procedures including but not limited to open, laparoscopic, single incision, natural orifice, endoscopic, non-invasive, various combination of, various interventional spaces for catheter-based procedures of the vascular, cardiovascular pulmonary and/or neurocranial-related spaces, cosmetics/aesthetics, metabolic (e.g., type 2 diabetes), plastic and reconstructive, ocular and ophthalmology, orthopedic, gynecology and men's health, and other systems, devices and methods of treating diseased, injured, undesired, or healthy tissues, organs or cells.

Systems and methods are also provided for improving treatment patterns within tissue that can reduce treatment time, improve efficacy, and reduce the amount of energy and prefocal tissue heating delivered to patients.

### Use Environments

The disclosed system, methods of use, and use of the system, may be conducted in a plethora of environments and settings, with or without various support systems such as anesthesia, including but not limited to, procedure suites, operating rooms, hybrid rooms, in and out-patient settings, ambulatory settings, imaging centers, radiology, radiation therapy, oncology, surgical and/or any medical center, as well as physician offices, mobile healthcare centers or systems, automobiles and related vehicles (e.g., van), aero and marine transportation vehicles such as planes and ships, and/or any structure capable of providing temporary procedure support (e.g., tent). In some cases, systems and/or sub-systems disclosed herein may also be provided as integrated features into other environments, for example, the direct integration of the histotripsy Therapy sub-system into a MRI scanner or patient surface/bed, wherein at a minimum the therapy generator and transducer are integral to such, and in other cases wherein the histotripsy configuration further includes a robotic positioning system, which also may be integral to a scanner or bed centered design.

### Coupling

Systems may comprise a variety of Coupling sub-system embodiments, of which are enabled and configured to allow acoustic coupling to the patient to afford effective acoustic access for ultrasound visualization and acoustic cavitation/histotripsy (e.g., provide acoustic window and medium between the transducer(s) and patient, and support of). These may include different form factors of such, including open and enclosed device solutions, and some arrangements which may be configured to allow dynamic control over the acoustic medium (e.g., temperature, dissolved gas content, level of particulate filtration, sterility, volume, composition, etc.). Such dynamic control components may be directly integrated to the system (within the Cart), or may be in temporary/intermittent or continuous communication with the system, but externally situated in a separate device and/or cart.

The Coupling sub-system typically comprises, at a minimum, coupling medium (e.g., degassed water or water solutions), a reservoir/container to contain said coupling medium, and a support structure (including interfaces to other surfaces or devices). In most embodiments, the coupling medium is water, and wherein the water may be conditioned before or during the procedure (e.g., chilled, degassed, filtered, etc.). Various conditioning parameters may be employed based on the configuration of the system and its intended use/application.

The reservoir or medium container may be formed and shaped to various sizes and shapes, and to adapt/conform to the patient, allow the therapy transducer to engage/access and work within the acoustic medium, per defined and required working space (minimum volume of medium to allow the therapy transducer to be positioned and/or move through one or more treatment positions or patterns, and at various standoffs or depths from the patient, etc.), and wherein said reservoir or medium container may also mechanically support the load, and distribution of the load, through the use of a mechanical and/or electromechanical support structure. As a representative example, this may include a support frame. The container may be of various shapes, sizes, curvatures, and dimensions, and may be comprised of a variety of materials compositions (single, multiple, composites, etc.), of which may vary throughout. In some embodiments, it may comprise features such as films, drapes, membranes, bellows, etc. that may be insertable and removable, and/or fabricated within, of which may be used to conform to the patient and assist in confining/containing the medium within the container. It may further contain various sensors (e.g., volume/fill level), drains (e.g., inlet/outlet), lighting (e.g., LEDs), markings (e.g., fill lines, set up orientations, etc.), text (e.g., labeling), etc.

In one embodiment, the reservoir or medium container contains a scalable frame, of which a membrane and/or film may be positioned within, to afford a conformable means of contacting the reservoir (later comprising the treatment head/therapy transducer) as an interface to the patient, that further provides a barrier to the medium (e.g., water) between the patient and therapy transducer). In other embodiments, the membrane and/or film may comprise an opening, the patient contacting edge of which affords a fluid/mechanical seal to the patient, but in contrast allows medium communication directly with the patient (e.g., direct degassed water interface with patient). The superstructure of the reservoir or medium container in both these examples may further afford the proximal portion of the structure (e.g., top) to be open or enclosed (e.g., to prevent spillage or afford additional features).

Disclosed membranes may be comprised of various elastomers, viscoelastic polymers, thermoplastics, thermoplastic elastomers, thermoset polymers, silicones, urethanes, rigid/flexible co-polymers, block co-polymers, random block co-polymers, etc. Materials may be hydrophilic, hydrophobic, surface modified, coated, extracted, etc., and may also contain various additives to enhance performance, appearance or stability. In some embodiments, the thermoplastic elastomer may be styrene-ethylene-butylene-styrene (SEBS), or other like strong and flexible elastomers. The membrane form factor can be flat or pre-shaped prior to use. In other embodiments, the membrane could be inelastic (i.e., a convex shape) and pressed against the patient's skin to acoustically couple the transducer to the tissue. Systems and methods are further disclosed to control the level of contaminants (e.g., particulates, etc.) on the membrane to maintain the proper level of ultrasound coupling. Too many particulates or contaminants can cause scattering of the ultrasound waves. This can be achieved with removable films or coatings on the outer surfaces of the membrane to protect against contamination.

Said materials may be formed into useful membranes through molding, casting, spraying, ultrasonic spraying, extruding, and/or any other processing methodology that produces useful embodiments. They may be single use or reposable/reusable. They may be provided non-sterile, aseptically cleaned or sterile, where sterilization may comprise any known method, including but not limited to ethylene oxide, gamma, e-beam, autoclaving, steam, peroxide, plasma, chemical, etc. Membranes can be further configured with an outer molded or over molded frame to provide mechanical stability to the membrane during handling including assembly, set up and take down of the coupling sub-system. Various parameters of the membrane can be optimized for this method of use, including thickness, thickness profile, density, formulation (e.g., polymer molecular weight and copolymer ratios, additives, plasticizers, etc.), including optimizing specifically to maximize acoustic transmission properties, including minimizing impact to cavitation initiation threshold values, and/or ultrasound imaging artifacts, including but not limited to membrane reflections, as representative examples.

Open reservoirs or medium containers may comprise various methods of filling, including using pre-prepared medium or water, that may be delivered into the containers, in some cases to a defined specification of water (level of temperature, gas saturation, etc.), or they may comprise additional features integral to the design that allow filling and draining (e.g., ports, valves, hoses, tubing, fittings, bags, pumps, etc.). These features may be further configured into or to interface to other devices, including for example, a fluidics system. In some cases, the fluidics system may be an in-house medium preparation system in a hospital or care setting room, or conversely, a mobile cart-based system which can prepare and transport medium to and from the cart to the medium container, etc.

Enclosed iterations of the reservoir or medium container may comprise various features for sealing, in some embodiments scaling to a proximal/top portion or structure of a reservoir/container, or in other cases where sealing may comprise embodiments that seal to the transducer, or a feature on the transducer housings. Further, some embodiments may comprise the dynamic ability to control the volume of fluid within these designs, to minimize the potential for air bubbles or turbulence in said fluid and to allow for changes in the focal length to the target area without moving the transducer. As such, integrated features allowing fluid communication, and control of, may be provided (ability to provide/remove fluid on demand), including the ability to monitor and control various fluid parameters, some disclosed above. In order to provide this functionality, the overall system, and as part, the Coupling sub-system, may comprise a fluid conditioning system, which may contain various electromechanical devices, systems, power, sensing, computing, pumping, filtering and control systems, etc. The reservoir may also be configured to receive signals that cause it to deform or change shape in a specific and controlled manner to allow the target point to be adjusted without moving the transducer.

Coupling support systems may include various mechanical support devices to interface the reservoir/container and medium to the patient, and the workspace (e.g., bed, floor, etc.). In some embodiments, the support system comprises a mechanical arm with 3 or more degrees of freedom. Said arm may have a proximal interface with one or more locations (and features) of the bed, including but not limited to, the frame, rails, customized rails or inserts, as well as one or more distal locations of the reservoir or container. The arm may also be a feature implemented on one or more Carts, wherein Carts may be configured in various unlimited permutations, in some cases where a Cart only comprises the role of supporting and providing the disclosed support structure.

In some embodiments, the support structure and arm may be a robotically-enabled arm, implemented as a stand-alone Cart, or integrated into a Cart further comprising two or more system sub-systems, or where in the robotically-enabled arm is an arm of another robot, of interventional, surgical or other type, and may further comprise various user input features to actuate/control the robotic arm (e.g., positioning into/within coupling medium) and/or Coupling solution features (e.g., filling, draining, etc.). In some examples, the support structure robotic arm positional encoders may be used to coordinate the manipulation of the second arm (e.g. comprising the therapy transducer/treatment head), such as to position the therapy transducer to a desired/known location and pose within the coupling support structure.

Overall, significant unmet needs exist in interventional and surgical medical procedures today, including those procedures utilizing minimally invasive devices and approaches to treat disease and/or injury, and across various types of procedures where the unmet needs may be solved with entirely new medical procedures. Today's medical system capabilities are often limited by access, wherein a less or non-invasive approach would be preferred, or wherein today's tools aren't capable to deliver preferred/required tissue effects (e.g., operate around/through critical structures without serious injury), or where the physical set up of the systems makes certain procedure approaches less desirable or not possible, and where a combination of approaches, along with enhanced tissue effecting treatments, may enable entirely new procedures and approaches, not possible today.

In addition, specific needs exist for enabling histotripsy delivery, including robotic histotripsy delivery, wherein one or more histotripsy therapy transducers may be configured to acoustically couple to a patient, using a completely sealed approach (e.g., no acoustic medium communication with the patient's skin) and allowing the one or more histotripsy transducers to be moved within the coupling solution without impeding the motion/movement of the robotic arm or interfering/disturbing the coupling interface, which could affect the intended treatment and/or target location.

Disclosed herein are histotripsy acoustic and patient coupling systems and methods, to enable histotripsy therapy/treatment, as envisioned in any setting, from interventional suite, operating room, hybrid suites, imaging centers, medical centers, office settings, mobile treatment centers, and/or others, as non-limiting examples. The following disclosure further describes novel systems used to create, control, maintain, modify/enhance, monitor and setup/takedown acoustic and patient coupling systems, in a variety of approaches, methods, environments, architectures and work-flows. In general, the disclosed novel systems may allow for a coupling medium, in some examples degassed water, to be interfaced between a histotripsy therapy transducer and a patient, wherein the acoustic medium provides sufficient acoustic coupling to said patient, allowing the delivery of histotripsy pulses through a user desired treatment location (and volume), where the delivery may require physically moving the histotripsy therapy transducer within a defined work-space comprising the coupling medium, and also where the coupling system is configured to allow said movement of the therapy transducer (and positioning system, e.g., robot) freely and unencumbered from by the coupling support system (e.g., a frame or manifold holding the coupling medium).

### Coupling System and Sub-Systems/Components

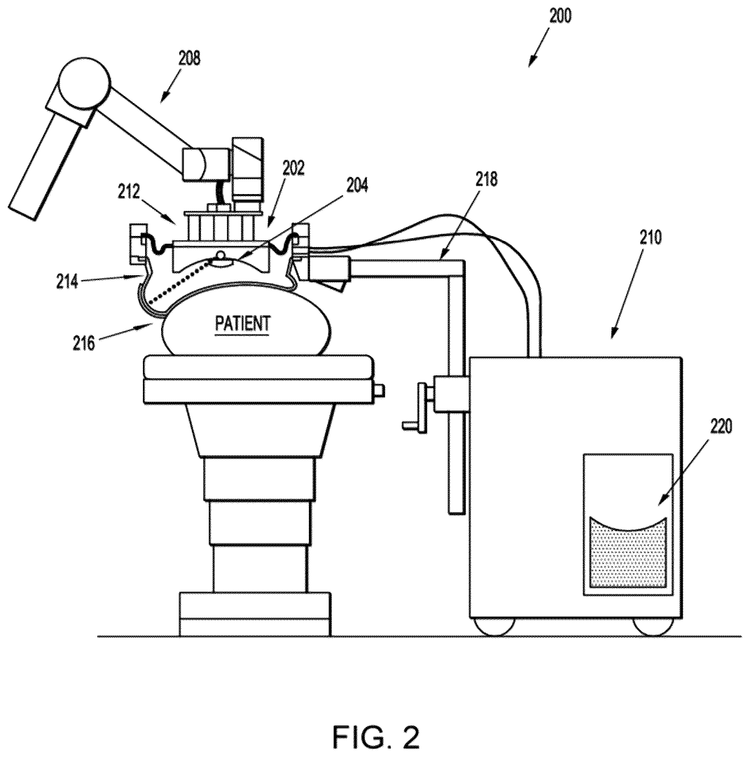

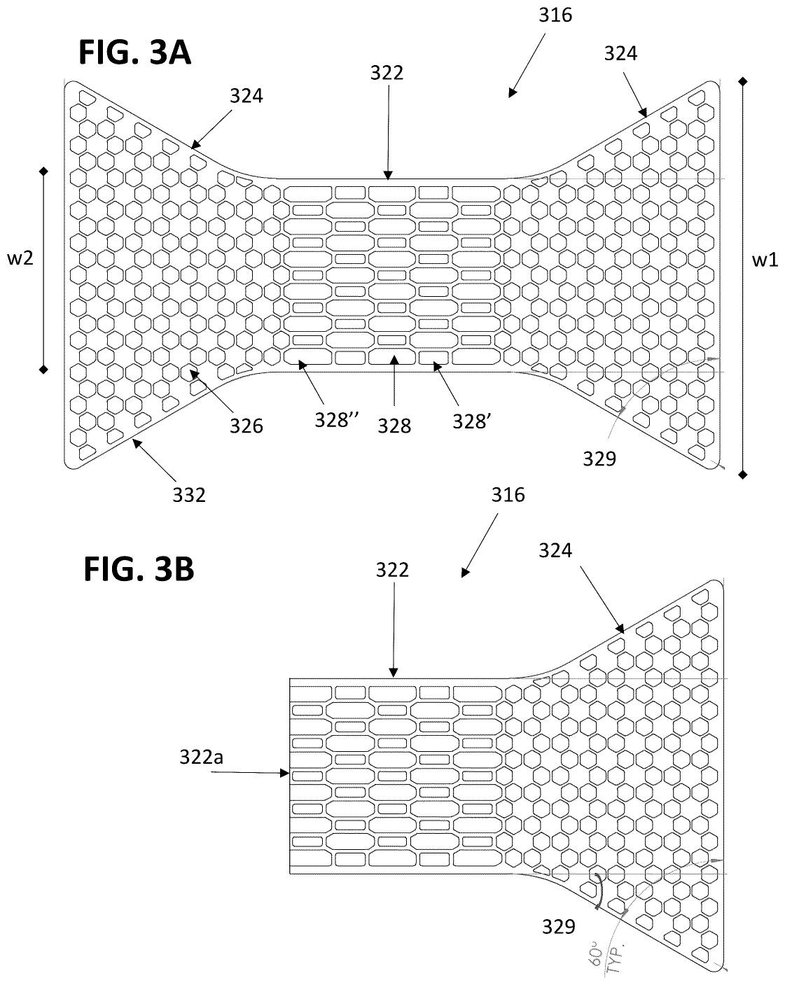

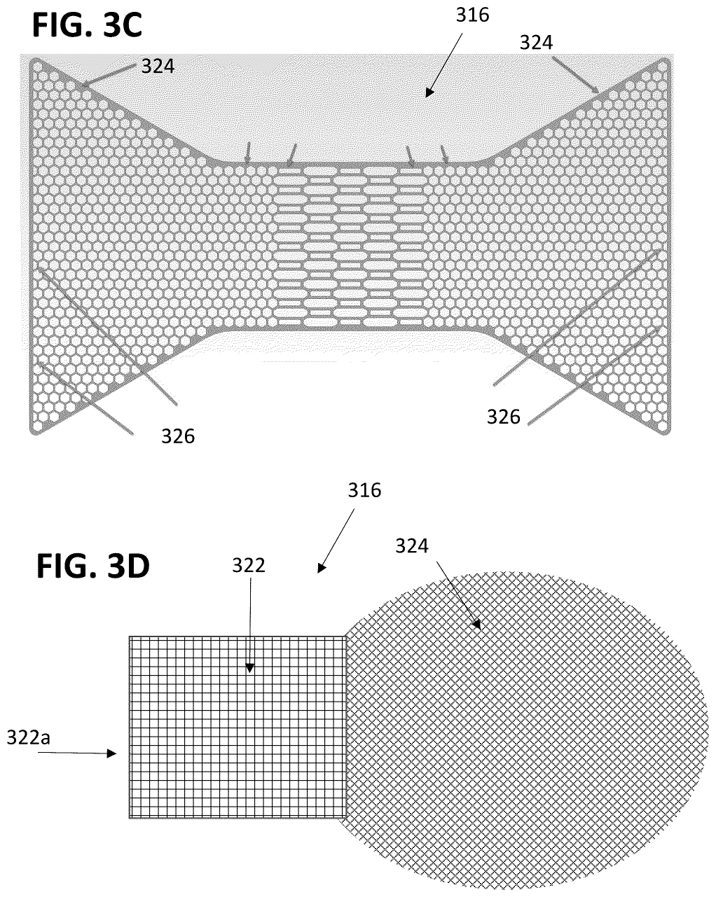

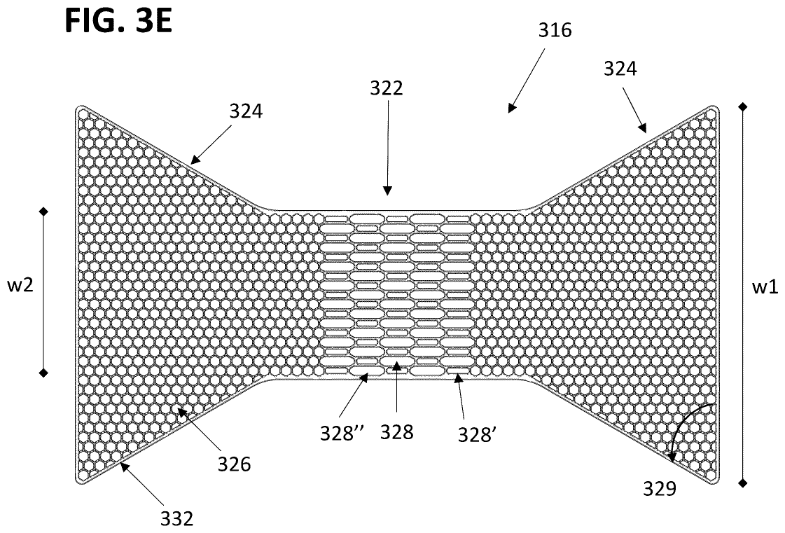

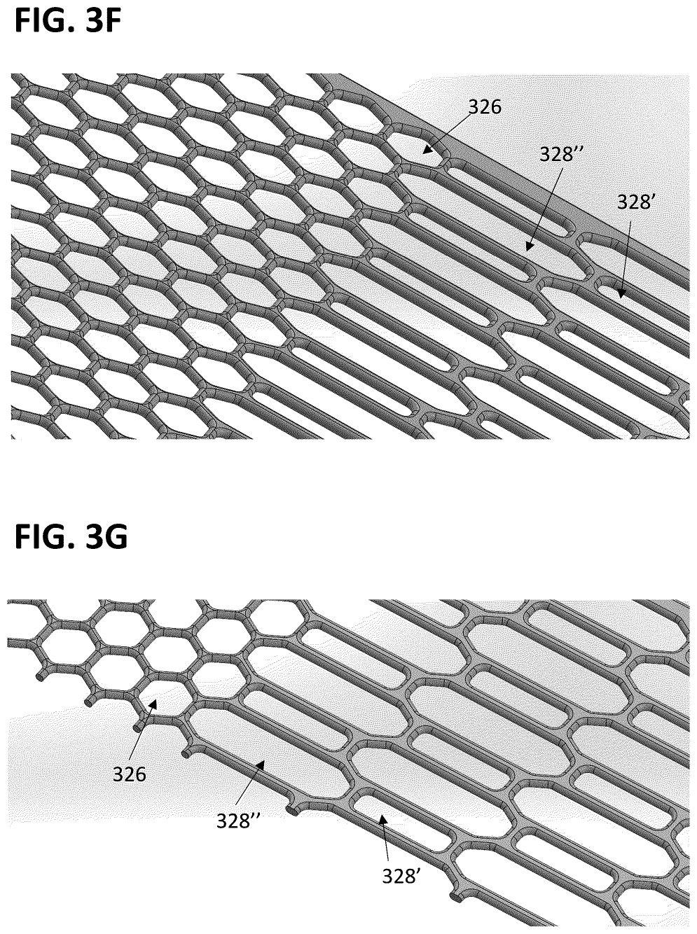

The disclosed histotripsy acoustic and patient coupling systems, in general, may comprise one or more of the following sub-systems and components, an example of which is depicted in at least FIGS. 2-3, including but not limited to 1) a membrane/barrier film to provide an enclosed, sealed and conformal patient coupling and histotripsy system interface, 2) a frame and assembly to retain the membrane and provide sufficient work and head space for a histotripsy therapy transducers required range of motion (x, y and z, pitch, roll and yaw), 3) a sufficient volume of ultrasound medium to afford acoustic coupling and interfaces to a histotripsy therapy transducer and robotic arm, 4) one or more mechanical support arms to allow placement, positioning and load support of the frame, assembly and medium and 5) a fluidics system to prepare, provide and remove ultrasound medium(s) from the frame and assembly.

In some embodiments, the coupling system may be fully sealed, and in other embodiments and configurations, it may be partially open to afford immediate access (physical and/or visual).

The acoustic and patient coupling systems and sub-systems may further comprise various features and functionality, and associated work-flows, and may also be configured in a variety of ways to enable histotripsy procedures as detailed below.

FIG. 2 illustrates one embodiment of a histotripsy therapy and imaging system 200, including a coupling assembly 212. As described above, a histotripsy therapy and imaging system can include a therapy transducer 202, an imaging system 204, a robotic positioning arm 208, and a fluidics cart 210. The robotic positioning arm may be attached to a therapy cart, such as cart 110 from FIG. 1A.

The therapy and/or imaging transducers can be housed in a coupling assembly 212 which can further include a coupling membrane 214 and a membrane constraint 216 configured to prevent the membrane from expanding too far from the transducer. The coupling membrane can be filled with an acoustic coupling medium such as a fluid or a gel. The membrane constraint can be, for example, a semi-rigid or rigid material as compared to the membrane, and configured to restrict expansion/movement of the membrane. In some embodiments, the membrane constraint is not used, and the elasticity and tensile strength of the membrane prevent over expansion. The coupling membrane can be a mineral-oil infused SEBS membrane to prevent direct fluid contact with the patient's skin. In the illustrated embodiment, the coupling assembly 212 is supported by a mechanical support arm 218 which can be load bearing in the x-y plane but allow for manual or automated z-axis adjustment. The mechanical support arm can be attached to the floor, the patient table, or the fluidics cart 210. The mechanical support is designed and configured to conform and hold the coupling membrane 214 in place against the patient's skin while still allowing movement of the therapy/imaging transducer relative to the patient and also relative to the coupling membrane 214 with the robotic positioning arm 208.

The fluidics cart 210 can include additional features, including a fluid tank 220, a cooling and degassing system, and a programmable control system. The fluidics cart is configured for external loading of the coupling membrane with automated control of fluidic sequences. Further details on the fluidics cart are provided below.

FIGS. 3A-3E illustrate embodiments of a membrane constraint 316 which can also be referred to herein as a constraint device. The constraint device 316 of the present disclosure comprises a mesh structure with open pores having varied geometrical shapes as described herein. In general, the constraint device is configured provide mechanical support to the membrane when the membrane is filled with a coupling medium/fluid. In particular, the constraint device may prevent the coupling membrane 214 from overexpanding or expanding too far away from the transducer while enabling the coupling membrane 214 to conform to the geometry of the patient. The constraint device may also minimize any forces applied against a patient and reduces any load transfer to the patient when the membrane is filled with the coupling medium/fluid. In contrast to the constraint of FIG. 2, the membrane constraint 316 of FIGS. 3A-3E is configured to include a portion or section that rests under the patient and is further configured to have one or more portions adapted and configured to attach or be coupled to the coupling assembly, frame of the coupling assembly, or operating table.

It should be noted that with regards to the embodiments described herein, FIGS. 3A, 3C, and 3E can generally include similar features expect when explicitly denoted, as with FIGS. 3B and 3D. Any aspect or combination of features or designs among the embodiments of FIGS. 3A-3E can be combined or individually selected for other similar embodiments of membrane constraints.

Referring to FIGS. 3A-3E, the constraint device 316 can include a patient portion 322 and one or more peripheral portions or sections 324 extending outward from and/or coupled to the patient portion 322. For example, the embodiment of FIGS. 3A and 3E shows a constraint device 316 with a patient portion 322 and a first and second peripheral portions 324 extending outward from opposite sides of the patient portion. However, the embodiment of FIGS. 3B and 3D show a membrane constraint 316 with a patient portion 322 and only one peripheral portion 324 extending from the patient portion. In some embodiments, the constraint device 316 comprises one or more peripheral portions, each peripheral portions being similar in size and shape to one another. Alternatively, one peripheral portion may be larger in size than the other. Still in other embodiments, each peripheral portion may have different shapes, depending on if the patient is right or left-facing, or side-lying with respect to the histotripsy system. Combinations of both size and shape for each peripheral portion are also envisioned. It should be noted that with the exception of the single peripheral portion, most other features remain similar to the other embodiments described and illustrated herein.

In some embodiments, the peripheral portion(s) 324 of the constraint device 316 can include a plurality of openings 326. Similarly, the patient portion 322 can also include a plurality of openings 328. In some examples, the openings 326 of the peripheral portion and openings 328 of the patient portion can be arranged to form a pattern in the peripheral and patient portions, respectively. In some embodiments, the pattern of openings in the peripheral portion(s) can be different than the pattern of openings in the patient portion. In other embodiments the patterns of the patient and peripheral portion openings can be the same. In general, the openings in the peripheral portion(s) are configured to engage with, attach to, or interface with the coupling assembly. For example, the coupling assembly can include an exterior frame which may include hooks, tabs, buttons, or other attachment features which can be connected or attached to the constraint device 316 with the openings of the peripheral portion(s).

In one embodiment, as shown in FIGS. 3A-3B and 3E, the pattern of openings 326 in the peripheral portion(s) can comprise a honeycomb pattern or arrangement. In some implementations, the individual openings can include hexagonal shapes. In particular embodiments, only hexagonal geometrical openings may be employed in the peripheral portion. The hexagonal openings can be closely spaced together and can have the same dimensions. Smaller hexagonal openings may assist with load sharing of the fluid-filled membrane of the coupling assembly when the constraint is positioned against the membrane. In particular, at least two, and in some embodiments, more than three distinct geometrical shapes may be employed in the constraint.

Patterns including shapes which have at least one interior angle may be preferable, such as triangles, squares, rectangles, pentagons, trapezoids, parallelograms, and the like. However, any other shapes can be used for the openings, including circles, ovals, and other shapes including radius of curvature, etc. Combinations of shapes are also envisioned. In some embodiments, the openings within the peripheral portion(s) can have different sizes and dimensions. For example, the honeycomb pattern shown in FIG. 3A includes a plurality of smaller hexagonal openings disposed around a singular larger hexagonal opening. However, in FIG. 3C, the openings within the peripheral portion(s) can have similar or the same sizes throughout the peripheral portion(s). The peripheral patient portion is optimized for load distribution and support along the coronal plane of the patient. The openings of the peripheral portion comprise engagement features which can be removably connected to the attachment features of the ultrasound coupling assembly. In particular, a diameter of the honeycomb openings may be sized to be received by a corresponding attachment feature on the coupling assembly frame. In some embodiments, the openings of the constraint device may be smaller than the attachment features of the coupling assembly, but the compliant nature of the constraint device may allow the openings to stretch over and conform to the attachment features.

A central portion/patient portion 322 of the membrane constraint can include openings of a different pattern and geometrical shape than the openings in the peripheral portion(s). In the embodiments of FIGS. 3A and 3E, the central portion may include a pattern of two or more different geometrical openings including, for example, a rectangular opening 328′ and an elongated hexagon or oval opening 328″. The pattern is illustrated as alternating ABAB (e.g., 328′, 328″, 328′, 328″), however other patterns are also envisioned such as AABB, ABCACB, AABBCC, and the like. It is also within the scope of this invention that the central patient portion may include only one geometrical shape, which may be different than the hexagon pattern/shape of the peripheral portion. It should be noted that the interior corners of any portion of the openings may be rounded, as illustrated in FIG. 3E. As illustrated, the constraint includes two or more different shaped geometrical openings. In embodiments, the constraint includes three or more different shaped geometrical openings. Specifically, more, smaller dimensioned geometrical shaped openings (e.g., smaller hexagons) may be disposed adjacent to or in proximity to the coupling assembly. Larger, elongated openings of the central portion may be positioned in contact with the patient or underneath the patient, such that the elongated direction of the openings is generally perpendicular to a longitudinal axis of the patient, such that the constraint is configured to elongate along a transverse axis of the patient.

FIGS. 3F and 3G show close-up views of the patterns of openings in the peripheral and patient portions of the constraint device, including openings 326 in the peripheral portion(s) and openings 328′ and 328″ in the central or patient portion of the constraint device.

As described above, in some embodiments the patient portion 322 can include a different pattern of openings than the openings of the peripheral portion(s). In general, the openings of the peripheral portion(s) are optimized and configured for attachment to attachment features of the coupling assembly, whereas the openings of the patient portion are typically not attached to the coupling assembly. Instead, the openings of the patient portion can be optimized and configured to prioritize patient comfort since the patient's weight is placed on the patient portion and the patient remains laying on the patient portion during the entirety of the histotripsy procedure. In some embodiments, the pattern of openings in the patient portion can be configured to reduce hot spots or pressure points. In other embodiments, the patient portion may not include any openings but instead may just be a solid or flat portion of the compliant material.

The peripheral portion(s) 324 of the constraint device are configured to be attached or coupled to the coupling assembly or coupling solution of the ultrasound therapy system. In some examples, as described herein, the coupling assembly can include an ultrasound coupling assembly (e.g., coupling assembly 212 in FIG. 2) and a coupling membrane (e.g., coupling membrane 214 in FIG. 2). The coupling assembly can be filled with an ultrasound coupling medium, such as degassed saline or water, enabling the membrane to expand such that the therapy head of the histotripsy system can be received within the fluid-filled coupling assembly and be acoustically coupled to the patient. The coupling membrane itself can comprise flexible and elastomeric materials, which can result in stretching or expansion of the coupling membrane when the coupling assembly is filled with the ultrasound coupling medium. With no constraint device in place, the volume of coupling medium (e.g., 10-12L or more) within the coupling assembly can cause over-expansion of the membrane which can result an insufficient workspace to allow for movement of the therapy head according to a treatment plan, and/or the inability to properly acoustically couple the therapy head with the patient.

Still referring to FIGS. 3A-3D and 3E, in some embodiments the peripheral portion(s) 324 can have a larger width w1 than a width w2 of the patient portion. As shown, the peripheral portions can flare out as they extend away from the patient portion. For example, the peripheral portion(s) may start at a width w2 where they meet or join the patient portion, and may flare or extend out to a width w1 that is greater than the width w2.

In some embodiments, this expanding width may be uniform, such that the sides 332 of the peripheral portion(s) maintain a straight edge. However, it should be understood that sides 332 may be curved, angled or jagged or other shapes that may better facilitate attachment of the constraint to the coupling assembly. For example, the sides 332 may be curved inwards or outwards to better conform to a patient when the peripheral portion(s) are wrapped around the patient and attached to the coupling assembly. In the example of FIG. 3A and FIG. 3E, the peripheral portion(s) 324 may flare out at an angle 329. For example, the angle 329 may be as low as 10-15 degrees and go up to 75-90 degrees. In the illustrated example, the angle 329 can be approximately 20-50 degrees, and preferably about 35 degrees. The uniform flaring of the membrane constraint on each of the peripheral portions of FIG. 3A gives the constraint a shape resembling a bow tie. It is also envisioned that more than one angle 329 or different angles on each peripheral portion can be employed. Alternatively, a smooth transition to the peripheral portions, such as an arc or curve is also envisioned.

An alternate embodiment is illustrated in FIGS. 3B and 3D, including a one-sided, flanged configuration. For example, the embodiment of FIG. 3D shows a constraint device 316 with a first, patient-contacting portion 322 and a second, flared portion 324 extending outward from the first, patient-contacting portion 322. In this particular embodiment, the first, patient contacting portion 322 may be configured to include attachment features which attach an exterior portion 322a of the patient-contacting portion 322 to the operating table, C-arm or other mechanically stable structure. Specifically, exterior portion 322a is able to attach directly to, for example the operating table by attaching the elongated honeycomb structures to an attachment feature found on the operating table. In particular, the one-sided flanged portion configuration could be desirable, for example, for particular patients where a treatment area could be accessed easier if only one flanged portion is employed. A one-sided flanged constraint device could also be useful for a side-lying patient or perhaps for a smaller patient.

With regards to FIG. 3D. an exterior of the patient contacting portion 322a or patient contacting portion 322 may also be configured to attach to the operating table. The exterior patient contacting portion 322a may attach to hooks or other features which extend outward from the operating table to receive an opening in the constraint device 316. More than one opening in the constraint device 316 may be hooked or attached to the table to provide enhanced security. Further, more than one opening in the constraint device 316 may be attached to the same hook. The interaction or attachment of the constraint device openings to a hook or other attachment feature are configured for reversable attachment to one another.

Further, the constraint device may also be configured to accommodate adult and pediatric patients and may be provided in small, medium, large and extra-large sizes. Alternatively, it is envisioned that the constraint device may be provided in only one size and is configured to accommodate patients of all shapes and sizes. The repeating pattern of the peripheral portion openings (which may be hexagonal, honeycomb shaped, etc.) enables the constraint to be adjustable and repositionable when attaching to the coupling assembly. For example, in a larger patient, the constraint may be attached to the coupling assembly, using more of the hexagonal openings disposed closer to an exterior edge of the peripheral portions. This enables more extension of the coupling assembly membrane, such that the membrane can be shaped to conform to a larger patient, while having the mechanical support from the constraint device, while maintaining acoustic coupling between the transducer and patient. In another example, for a smaller patient, the constraint device may be attached to the coupling assembly at a more interior or central portion of the peripheral flared portion (likely label as well). This enables, the coupling assembly membrane to expand or extend less, such that the membrane can be shaped to conform to a smaller patient, while having the mechanical support from the constraint device, while maintaining acoustic coupling between the transducer and the patient.

In general, the patient portion 322 of the constraint device 316 is configured to be positioned under a patient undergoing a histotripsy or other therapeutic ultrasound procedure. For example, the constraint device can be placed on a medical or operating table, and the patient can then be positioned on top of the patient-contacting portion 322. In other words, the constraint device 316 may be positioned between the patient and the operating/procedure table. The weight of the patient on top of the patient portion is typically sufficient to hold the membrane constraint in place during a histotripsy procedure so to enable acoustic coupling between the therapy transducer and the patient. However, in some embodiments, the patient-contacting portion 322 can be temporarily or permanently affixed to the medical or operating table as will be described in more detail below.

In some implementations, the membrane constraint can comprise a flexible or compliant material. The constraint can be an elastomer such as silicon or rubber or other similar materials. In particular, the constraint may be a silicone elastomer having a Shore durometer of 50A +/−5A. Generally, the membrane constraint can have some compliance or elasticity. However, in order to effectively constrain the coupling membrane when the coupling assembly 212 is filled with a coupling medium, the membrane constraint should be less flexible or compliant than the coupling membrane (e.g., be stiffer than the membrane).

The constraint device described herein can be a monolithic structure. In embodiments, the constraint device may be manufactured from a single sheet of polymer and created using a die-cut process. In other embodiments, the constraint device may be manufactured using injection molding, or 3D printed. Specifically, injection molding the constraint enables the constraint to have a radiused exterior, with curved exterior portions. The constraint device may be provided as a transparent or translucent material such that surgical field visibility is optimal.

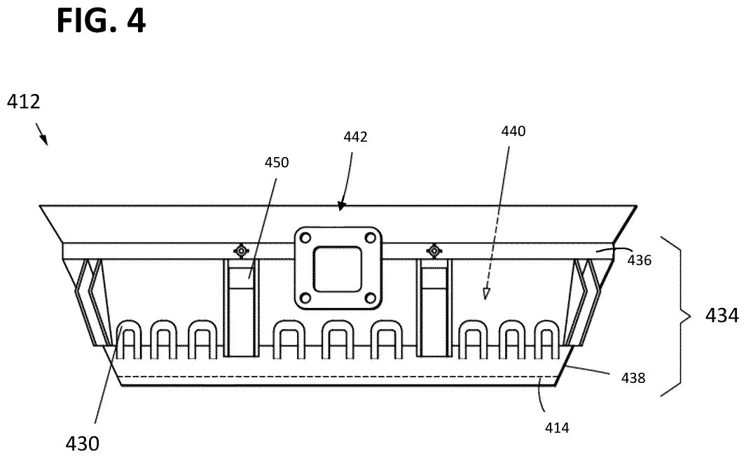

FIG. 4 provides an example of a coupling assembly 412 that includes a frame body 434 having an upper frame body 436 and a lower frame body 438. A coupling membrane 414 can be included in the coupling assembly. In some embodiments, the coupling membrane is positioned between the lower frame body and the upper frame body. In other embodiments, the membrane is attached or coupled to the lower frame body, which is attachable to the upper frame body with latches or clamps 450. Frame cavity 440 is configured to be filled with an acoustic coupling medium prior to a histotripsy procedure. The coupling assembly, including optionally the upper frame body and/or lower frame body, may include a plurality of attachment features 430 configured to be attached to or removably coupled with a constraint device as discussed herein. A histotripsy or ultrasound therapy transducer can then be inserted into the coupling assembly 412 through top portion or opening 442 of the coupling assembly for acoustic coupling to the patient.

As shown, the coupling assembly can include a plurality of attachment features. In some embodiments, the attachment features 430 can be disposed around a perimeter of the coupling assembly. The attachment features 430 protrude from an exterior surface of the coupling assembly. The attachment features 430 extend outward from the coupling assembly to receive a portion of the peripheral portion thereon. In other embodiments, the attachment features may be disposed only in selected portions of the coupling assembly where attachment to the membrane constraint is desired. In general, a larger number of attachment features around the entirety of the perimeter of the coupling assembly provides greater flexibility in attachment points between the coupling assembly and the membrane constraint. The attachment features can attach to one or more openings in the constraint device. For example, if further security of the membrane or device is required, multiple openings in the peripheral portions can be attached to a single attachment feature.

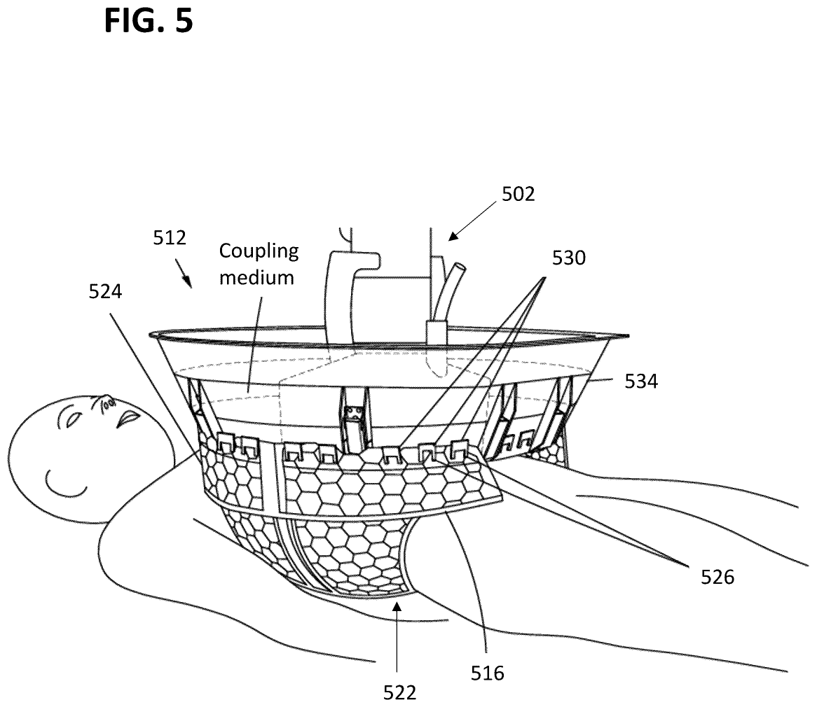

FIG. 5 illustrates one embodiment of a constraint device 516 coupled to a coupling assembly 512 having a frame body 534 with a patient laying on the patient portion 522 of the constraint device (e.g., patient portion 322 described above). Particularly, at least some of the attachment features 530 of the coupling assembly are positioned through some of the openings 526 of the peripheral portion 524 of the constraint device 516 to attach (i.e., hang, suspend, connect) the constraint device 516 to the coupling assembly. As described above, the coupling membrane can be expanded as the coupling assembly 512 is filled with an ultrasound coupling medium for acoustic coupling to an ultrasound transducer array 502. In this example, the stiffness or flexibility of the membrane constraint relative to the stiffness or flexibility of the coupling membrane prevents overexpansion of the coupling membrane. In particular, the stiffness or flexibility or the membrane constraint is less flexible and has a lower % elongation compared to the coupling membrane.

The flared, angled, or curved edges of the peripheral portions 524 of the constraint device allow for the constraint device to be attached at various points along the coupling assembly while also maintaining a close fit to the contours of the patient. Depending on the location of the target tissue, the patient may be positioned on his or her back, stomach, or side. The flared peripheral portions provide flexibility in how the constraint is attached to the coupling assembly, enabling a variety patient positions, treatment head locations, and coupling assembly locations.

### Coupling Kit

The present disclosure further describes a coupling kit for use with histotripsy or ultrasound cavitation therapy. The coupling kit includes one or more parts suitable for therapeutically connecting a patient to a treatment device for histotripsy or ultrasound cavitation, e.g., a transducer. The coupling kit may be designed to be a disposable and/or single-use kit. However, because the coupling kit does not have to be sterile when used, in some embodiments, the coupling kit or at least some parts of the coupling kits described herein may be reusable. The coupling kit includes one or more of a coupling membrane, a coupling membrane constraint, a coupling band, a bubble removal tool, an inspection mirror, a container of oil, or any combination thereof.

In some embodiments, the coupling kit includes a membrane as described herein (with reference to at least FIG. 2). Generally, the membrane being a malleable, impermeable barrier that extends from the UMC coupling assembly to form an acoustically (and visually) transparent reservoir that contours to the patient and holds ultrasound medium. In some embodiments, the coupling kit includes a membrane as described herein and one or more of a membrane constraint, a coupling band, a bubble removal tool, a mirror, a container of oil, or any combination thereof.

In some embodiments, the coupling kit includes a membrane constraint as described herein (with reference to at least FIGS. 2-3D). Generally, the membrane constraint (or cradle) being an elastic webbing that extends from the coupling assembly and is positioned around the patient (FIG. 5) to prevent the coupling membrane from excessively bulging or shifting on the patient. In some embodiments, the coupling kit includes a membrane constraint as described herein and one or more of a coupling membrane, a coupling band, a bubble removal tool, a mirror, a container of oil, or any combination thereof.

In some embodiments, the coupling kit includes a membrane and a membrane constraint as described herein. In some embodiments, the coupling kit includes a coupling membrane and a membrane constraint as described herein and one or more of a coupling band, a bubble removal tool, a mirror, a container of oil, or any combination thereof.

In some embodiments, the coupling kit includes a container of oil. The container may be any container suitable for delivering the oil to an exterior of the patient. In some embodiments, the container is a bottle, such as a squeeze bottle, pour bottle, or spray bottle suitable for dispensing the oil to an exterior of the patient and ultimately between the exterior of the patient and the coupling membrane. One non-limiting example of a suitable oil includes castor oil. But other oils are also envisioned. The oil, and particularly castor oil, is applied to the patient's exterior to acoustically couple the patient membrane with the patient's skin, which helps fill in air pockets therebetween prior to the application of the oil and helps prevent their formation.

In some embodiments, the coupling kit includes a coupling band, and particularly in kits including a coupling membrane and/or a container of oil. The coupling band is designed to remove any air bubbles or microbubbles in the oil after the coupling membrane is applied to the exterior of the patient. Further, the coupling band/tube may assist in distributing oil across a surface of the patient. As may be noted hereinabove, air bubbles or microbubbles in the oil may distort and/or interfere with the therapeutic effects of histotripsy or ultrasound cavitation therapy.

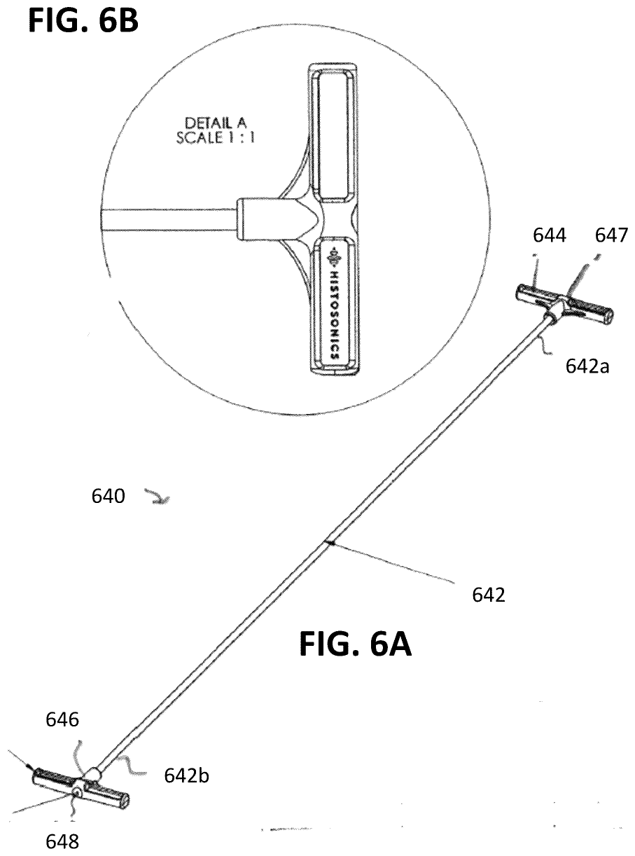

As depicted in FIGS. 6A-6B, the coupling band 640 includes a flexible tube 642, particularly but not limited to a flexible hollow tube, extending between a first end portion 642a and a second end portion 642b, a first handle 644 secured to the first end portion 642a of the tube 642 and a second handle 646 secured to the second end portion 642b of the tube 642. In some embodiments, at least one, if not both, of the first handle 644 or the second handle 646 is a generally T-shaped handle. In some embodiments, the first handle 644 is a first generally T-shaped handle secured to the first end portion 642a of the flexible tube 642 with a first T-shaped cap 647 and the second handle 646 is a second generally T-shaped handle secured to the second end portion 642b of the tube with a second T-shaped cap 648. The flexible tube of the coupling band is configured to be pulled, via first and second handles, slowly between the patient and the coupling membrane to evenly distribute the coupling oil and remove any air bubbles or microbubbles.

In some embodiments, the coupling kit may also include a bubble removal tool. The bubble removal tool is designed to remove air bubbles and/or air pockets formed within the ultrasound medium. Air bubble and/or air pockets may most often form around the base of the imaging probe or transducer following introduction into the ultrasound medium. Such air bubbles and/or air pockets have a reflective, metallic appearance against the dark therapy transducer and can be visible using an inspection mirror as described herein. The bubble removal tool can be used to remove the air bubbles or pockets via suction.

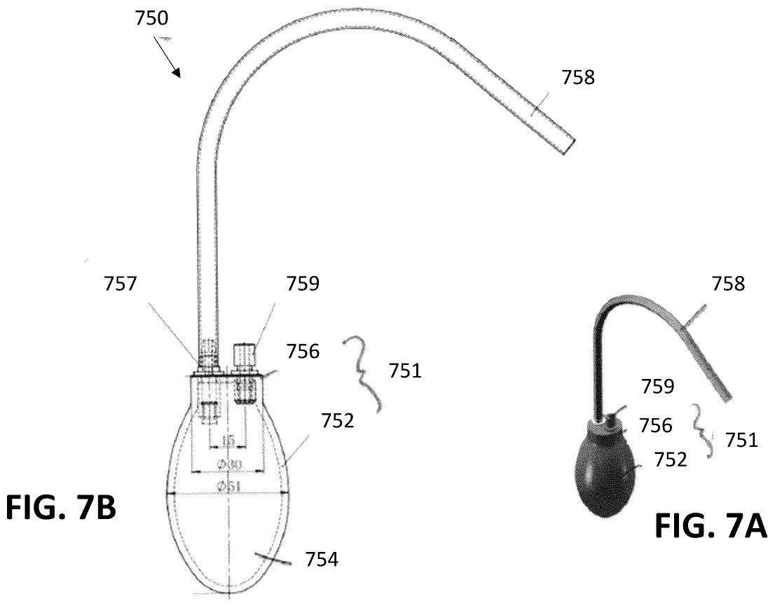

As shown in FIGS. 7A and 7B, in some embodiments, the bubble removal tool may be a suction bulb 750 including an inlet tube 758 coupled to an inlet valve 757 of a bulbous body 751. The bulbous body 751 includes a rounded base 752 and a flat top 756. The rounded base 752 defines a rounded bulb cavity 754 therein. The rounded base 752 is designed to be squeezed to cause suction to draw air bubbles or pockets into the inlet tube 758 (and/or bulb cavity 754). The rounded base (and/or any part of the suction bulb 750) may be made of squeezable material, including but not limited to, polyvinyl chloride. The squeezable material may display a Shore A hardness ranging from about 25 to 75 durometers, and in some embodiments, may display a Shore A hardness of about 50 durometers.

The bulbous body 751, and particularly the flat top 756 of the bulbous body 751, may further include a release valve 759. The release valve 759 is designed to release any air (or medium) captured in the cavity 754 and/or reduce pressure inside the cavity 754 to allow for additional suction. The inlet tube 758 may also be curved, as shown.

In some embodiments, the coupling kit may also include an inspection mirror. The inspection mirror is designed to be placed in the ultrasound medium to check for air bubbles and/or pockets along the face of the submerged transducer. If air bubbles or pockets are present, removal can be performed by placing the inlet tube of the bubble removal tool into the air bubble or pocket and squeezing the bulbous body causing the bubbles or pockets to be removed from the medium and sucked into the bulb cavity. Care should be taken when introducing the inspection mirror into the medium not to introduce additional air bubbles.

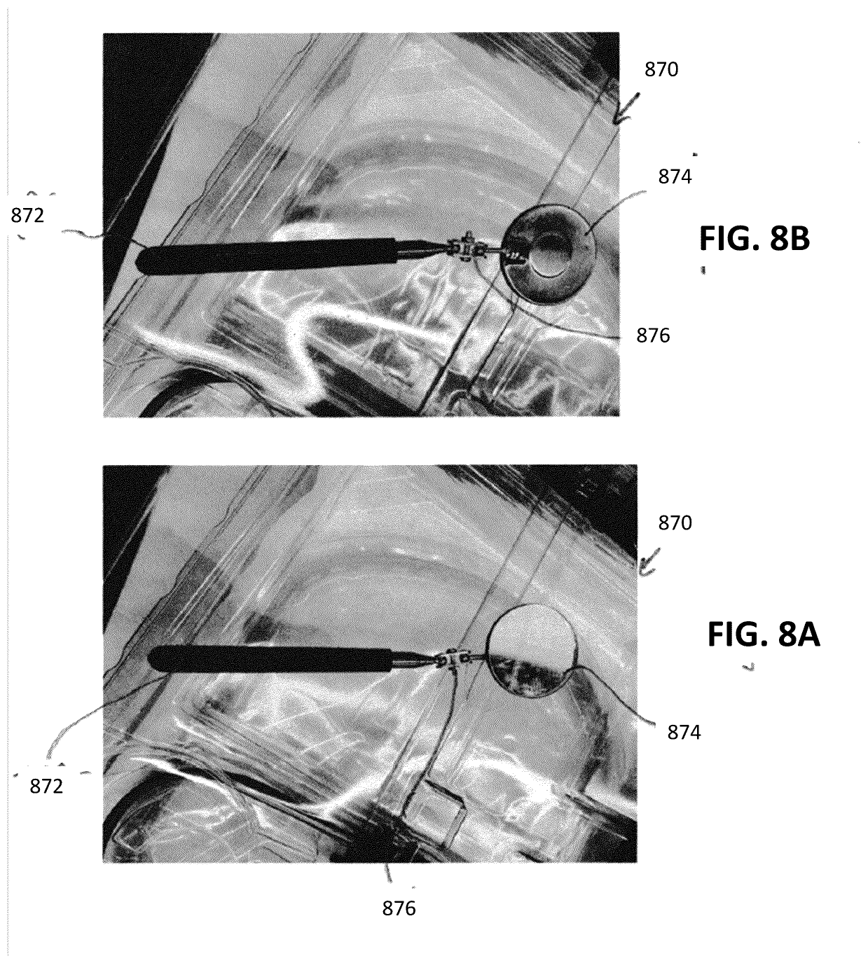

As shown in FIGS. 8A-8B, the inspection mirror 870 includes a mirror portion 874 pivotably attached via joint 876 to a handle portion 872. The handle portion 872 is designed to be manipulated by hand of a human being or by a robotic arm. Although the handle portion 872 is depicted as generally straight and the mirror portion 874 is generally circular, any suitable shape of the handle portion and/or mirror portion is envisioned.

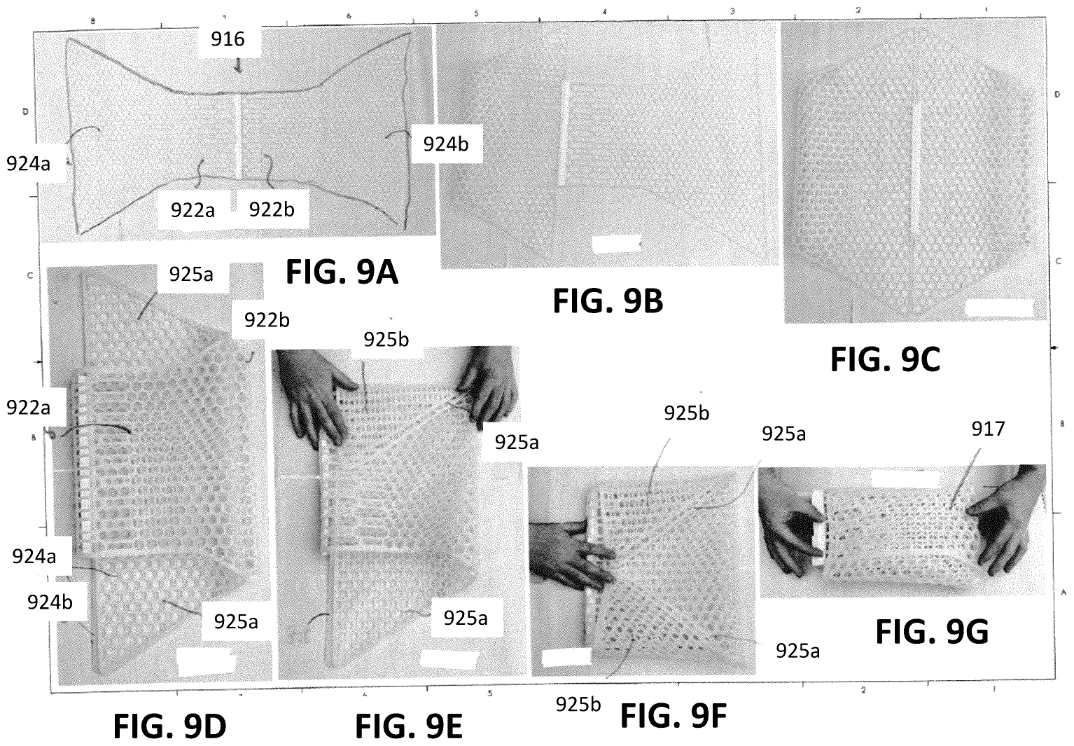

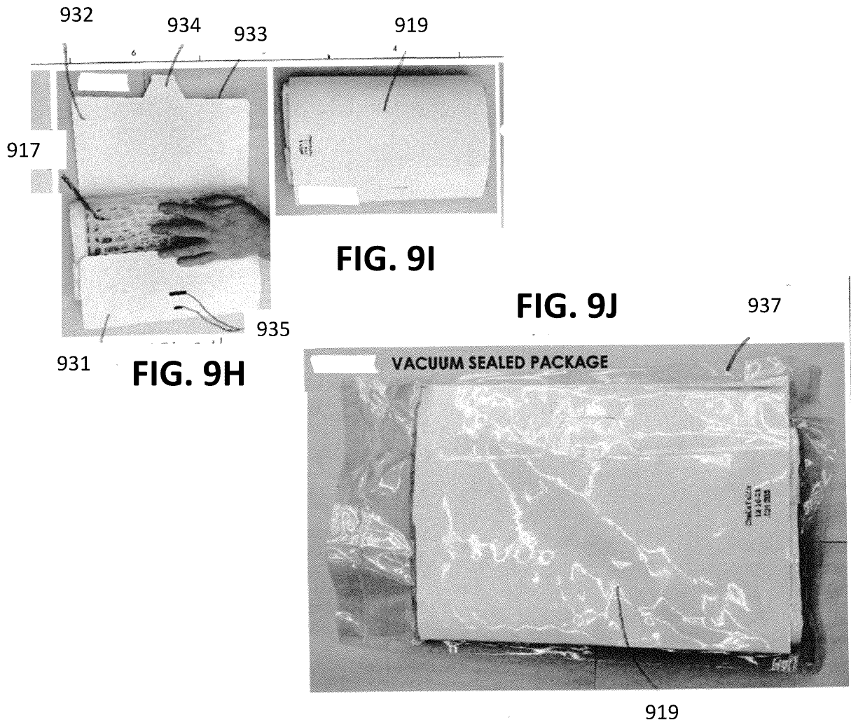

In some embodiments, the coupling kit may include a coupling membrane and/or a coupling membrane constraint in a folded and/or compacted manner. For example, as shown in FIGS. 9A-9J, a coupling membrane constraint 916 can be folded, wrapped in a folder, and sealed into a plastic package. Initially, as shown in FIGS. 9A-9C, a first peripheral portion 924a of a membrane constraint 916 is folded over a first half of a patient portion 922a and a second peripheral portion 924b of the membrane constraint 816 (opposite the first peripheral portion 924a) is folded over a second half of the patient portion 922b (opposite the first half 922a). Next, as shown in FIG. 9D, the first peripheral portion 824a (along with the first half of the patient portion 922a beneath the first peripheral portion 924a) is folded over the second peripheral portion 924b (with the second half of the patient portion 922b beneath the second peripheral portion 924b). Next, as shown in FIGS. 9E-9F, the outer flaps 925a, 925b of the first and second peripheral portions 924a, 924b, which are not sandwiched between the first and second halves of the patient portion 922a, 922b, are folded over the first half of the patient portion 922a. Next, as shown in FIG. 9G, the folded outer flaps 925a and 925b are folded again onto each other generally forming a folded constraint 917 having a width about half the width of the first or second patient portions. Next, as shown in FIGS. 9H-91, the folded constraint 917 can be wrapped by a folder 932, such as a tri-folder, which includes a folder tab 934 extending from one outer edge 933 and one or more tab openings 935 defined through a folder portion 931 opposite the folder tab 932. The folder tab 932 and tab opening 935 are designed to mating engage to close the folder 932 and/or wrap the folder 932 around the folded constraint 917. The folded and/or wrapped constraint 919 can then be placed within a plastic package 937 and sealed (FIG. 9J). The folded, wrapped, and/or packaged constraint can also be stored in a coupling kit multi-tray retainer as described in more detail hereinbelow.

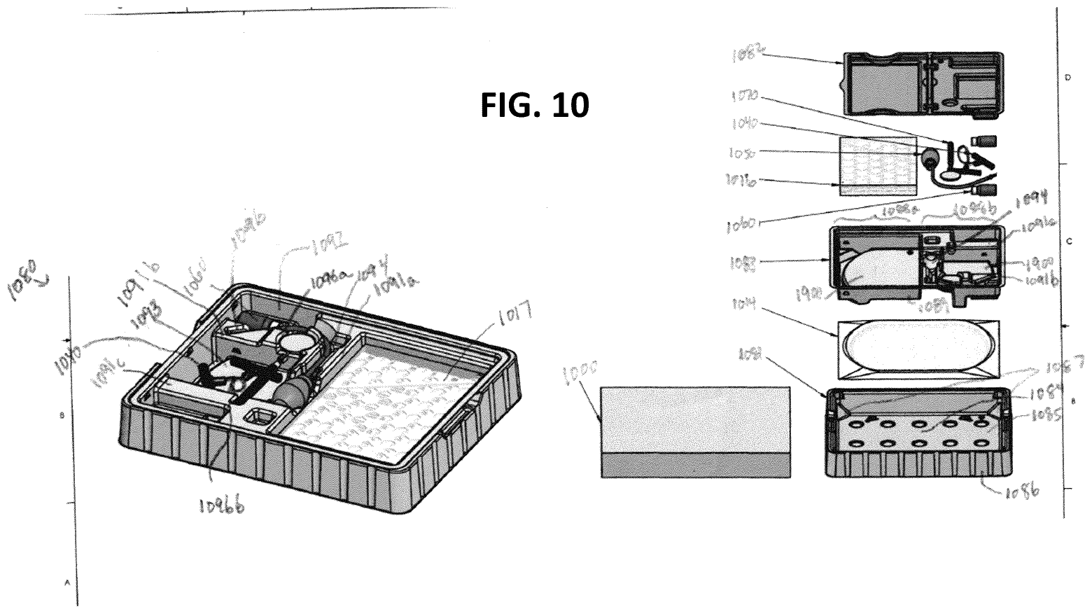

Turning to FIG. 10, a coupling kit as described herein can be stored and/or shipped in a multi-tray retainer 1080 designed to maintained within an outer carton and/or shipper box 1000. The inner multi-tray retainer 1080 may include a base tray 1081 and a cover tray 1082 with a middle tray 1083 designed to be positioned (i.e., stacked) between the base tray 1081 and cover tray 1082. Each of the base tray 1081, the cover tray 1082, and the middle tray 1083 may include one or more cavities defined therein to store a component of the coupling kit. For example, the base tray 1081 generally includes a base cavity 1084 defined therein to store a coupling membrane 1014 therein.

As further shown in FIG. 10, the base retainer 1080 includes a generally planar base floor 1085 having a base exterior sidewall 1086 extending generally vertical along an outer periphery of the base floor 1085. The base sidewall 1086 forms an exterior sidewall of the multi-tray retainer 1080. The base sidewall 1086 and the base floor 1085 define a base cavity 1084 configured to receive and/or maintain a membrane 1014 therein. In some embodiments, a triangular ramp 1087 may be positioned in at least one, if not all, corners of the base tray 1081 to aid in removing the membrane 1014 positioned therein. The base sidewall 1086 also has a sufficient height (and/or the base cavity 1084 has a sufficient depth) to further position the middle tray 1083 and cover tray 1082 over the membrane 1014 and within the base tray 1081, while including any additional parts of the coupling kit.

The middle tray 1083 is designed to sit within the base cavity 1084 (and/or base tray 1081) on top of the membrane 1014. The middle tray 1083 includes two generally equal-sized middle cavities (i.e., a first middle cavity 1088a and a second middle cavity 1088b) defined therein by middle exterior sidewall 1089 extending generally vertical along an outer periphery of the middle floor 1090 with a first inner sidewall 1091a connecting opposing sides of the middle exterior sidewall 1089. The first middle cavity 1088a is configured to store one or more constraint members 1016 therein, particularly in a folded configuration 1017.

The second middle cavity 1088b is configured to store therein one or more of a coupling band 1040, a bubble removal tool 1050, an inspection mirror 1070, a container of oil 1060, or any combination thereof. The second middle cavity 1088b may include the first inner wall 1091a, as well as a second and third inner sidewalls 1091b-c, defining a plurality of sub-cavities 1092, 1093, 1094. The first sub-cavity 1092 of the second middle cavity 1088b may be a corner sub-cavity configured to store a container of oil 1060 therein. The first sub-cavity 1092 defined between a corner of the exterior sidewall 1089 of the middle tray 1083, the second inner wall 1091b, and a portion of the first inner wall 1091a.

A second central sub-cavity 1093 is configured to store a coupling band 1040 therein. The second sub-cavity 1093 is defined between each of the first, second, and third inner walls 1901a-c and a portion of exterior sidewall 1089 of the middle tray 1083.

A third sub-cavity 1094 is configured to store a majority, if not all, of a bubble removal tool 1050 therein. The third sub-cavity 1094 is defined within a top surface of the first inner wall 1091a. In some embodiments, the bubble removal tool 1050 may include a curved inlet tube which extends out of the third sub-cavity 1094 across an upper open portion of the first sub-cavity 1092 and into a first depression 1095 defined within a top surface of the second inner wall 1093.

A pair of opposing indents 1096a, 1096b are defined within a top surface of the second and third inner walls 1091b-c such that opposing ends of an inspection mirror can be stored therein with a majority of the inspection mirror spanning across an upper open portion of the second sub-cavity 1093 and above the coupling band 1040. The first indent 1096a is generally circular to receive the mirror and the second indent 1096b is generally linear to receive the handle therein.

The cover tray 1082, like the middle tray 1083, is generally divided into two equal cavities and is designed to frictionally fit within the middle tray 1083 and/or base tray 1081 thereby covering the various components of the coupling kits stored and within the multi-tray retainer 1080. The multi-tray retainer 1080 including any combination of various components of a coupling kit can be placed in an outer box 1000 for shipping and/or delivery.

### Example 1

In one example, the treatment head is placed in contact with a system check container, which may be a fluidics cart. The system check container is filled with an ultrasound coupling medium. The treatment head is then lowered into the container until the ultrasound coupling medium reaches a depth line on the treatment head. Next, a user places the mirror at the bottom of the system check container and using the mirror inspects the treatment head for any air bubble or air pockets. If either of the air bubbles or air pockets are present, the user then removes them by placing the end of the bubble remover tool adjacent to the air bubbles/pockets and squeezing the bubble remover tool to suction out the air from the ultrasound coupling medium.

### Example 2

In another example, the coupling kit may be used in preparing the patient for a histotripsy procedure. The patient is placed on a procedure bed, with a constraint disposed underneath the patient. A handheld imagining probe may be used to assess the patient's anatomy in order to find the optimal position of the treatment head. Coupling oil is then applied to the patient's skin and spread over the abdomen as needed. The coupling band is then placed adjacent the constraint, with the coupling band placed lengthwise along the patient (handles closer to patient's head and feet, respectively). Once the coupling assembly has been filled with an ultrasound coupling medium, and the membrane is expanded against the patient's abdomen, the coupling band is then swiped or moved across the patient, with a user holding the handles. The coupling band is moved across the patient such that any residual bubbles disposed on a surface of the patient or coupling membrane are removed.

### Membranes/Barrier Films and Related Architectures

Membranes and barrier films may be composed of various biocompatible materials which allow conformal coupling to patient anatomy with minimal or no entrapped bubbles capable of interfering with ultrasound imaging and histotripsy therapy, and that are capable of providing a sealed barrier layer between said patient anatomy and the ultrasound medium, of which is contained within the work-space provided by the frame and assembly.

Membrane and barrier film materials may comprise flexible and elastomeric biocompatible materials/polymers, such as various thermoplastic and thermoset materials, as well as permanent or bioresorbable polymers. Additionally, the frame of the coupling assembly can also comprise the same materials. In some examples, the membrane may be rigid or semi-rigid polymers which are pre-shaped or flat.

### Ultrasound Medium

As previously described, the ultrasound medium may comprise any applicable medium capable of providing sufficient and useful acoustic coupling to allow histotripsy treatments and enable sufficient clinical imaging (e.g., ultrasound). Ultrasound mediums, as a part of this disclosure and system, may comprise, but are not limited to, various aqueous solutions/mediums, including mixtures with other co-soluble fluids, of which may have preferred or more preferred acoustic qualities, including ability to match speed of sound, etc. Example mediums may comprise degassed water and/or mixtures/co-solutions of degassed water and various alcohols, such as ethanol.

### Mechanical Support Arms and Arm Architectures

In order to support the acoustic and patient coupling system, including providing efficient and ergonomic work-flows for users, various designs and configurations of mechanical support arms (and arm architectures) may be employed. Support arms may be configured with a range of degrees of freedom, including but not limited to allowing, x, y, z, pitch, roll and yaw, as well additional interfacing features that may allow additional height adjustment or translation.

Arms may comprise a varied number and type of joints and segments. Typically, arms may comprise a minimum of 2 segments. In some configurations, arms may comprise 3 to 5 segments.

Arms are also be configured to interface proximally to a main support base or base interface (e.g., robot, table, table/bed rail, cart, floor mount, etc.) and distally to the frame/assembly and overall “coupling assembly” or “coupling solution”. This specific distal interface may further include features for controlling position/orientation of the frame/assembly, at the frame/assembly interface.

For example, in some embodiments, the arm/frame interface may comprise a ball joint wrist. In another example, the interface may include use of a gimbal wrist or an adjustable pitch and roll controlled wrist. These interfaces may be further employed with specific user interfaces and inputs, to assist with interacting with the various wrists, of which may include additional handles or knobs (as an unlimited example), to further enable positioning the coupling assembly. For example, a gimbal wrist may benefit from allowing the frame/assembly to have 3 degrees of freedom (independent of the arm degrees of freedom), including pitch, roll and yaw adjustments.

Support arms, configured with arm wrists, further interfaced with frames/assemblies, may comprise features such as brakes, including cable or electronic actuated brakes, and quick releases, which may interact with one or more axis, individually, or in groupings. They may also include electronic lift systems and base supports. In some embodiments, these lift systems/base supports are co-located with robot arm bases, wherein said robot arm is equipped with the histotripsy therapy transducer configured to fit/work within the enclosed coupling solution. In other embodiments, the support arm is located on a separate cart. In some cases, the separate cart may comprise a fluidics system or user console. In other embodiments, it is interfaced to a bed/table, including but not limited to a rail, side surface, and/or bed/table base. In other examples/embodiments, it's interfaced to a floor-based structure/footing, capable of managing weight and tipping requirements.

### Fluidics Systems, Control Systems and System Architectures

As a part of overall fluidics management, histotripsy systems including acoustic/patient coupling systems, may be configured to include an automated fluidics system, which primarily is responsible for providing a reservoir for preparation and use of coupling medium. The fluidics system may include the ability to degas, chill, monitor, adjust, dispense/fill, and retrieve/drain coupling medium to/from the coupling frame/assembly.

The fluidics system may include an emergency high flow rate system for rapid filling and draining of the coupling medium from the coupling assembly. The fluidics system may be configured to fill the coupling assembly with fluid on demand, or with predetermined fill amounts (e.g., automatic fill of a present volume of fluid such as IL, 3L 6L, 9L, etc.).

In some implementations, the fluidics system is configured to connect to or receive fluid from a fluid source such as tap water. The fluidics system can include a degas system or mechanism such as a degas membrane that can be configured to degas fluid as it flows from the fluid source into the fluid tank of the fluidics system. The degas system can be further configured to degas the fluid as it flows from the fluid tank to the coupling assembly. In some implementations, the fluid is degassed to a first degas threshold while the fluid tank is filled from the fluid source, and held at the first degas threshold. The fluidics system can then further degas the fluid as it is transferred from the fluid tank to the coupling assembly (e.g., to a second degas threshold).

In some embodiments, the fluidics system can be configured for a single use of the coupling medium, or alternatively, for re-use of the medium. In some embodiments, the fluidics system can implement positive air pressure or vacuum to carry out leak tests of the coupling assembly and membrane prior to filling with a coupling medium. Vacuum assist can also be used for removal of air from the coupling assembly during the filling process. The fluidics system can further include filters configured to prevent particulate contamination from reaching the coupling assembly.

The fluidics system may implemented in the form of a mobile fluidics cart. The cart may comprise an input tank, drain tank, degassing module, fill pump, drain pump, inert gas tank, air compressor, tubing/connectors/lines, electronic and manual controls systems and input devices, power supplies and one or more batteries. The cart in some cases may also comprise a system check vessel/reservoir for evaluating histotripsy system performance and related system diagnostics (configured to accommodate a required water volume and work-space for a therapy transducer).

The cart may be powered through standard electrical service/connectors, as well as with a battery to allow for portable or off-grid use. The battery may also provide emergency power. The cart may also comprise a nitrogen tank and/or air compressor (not shown) for allowing blow down of the main/drain tubing to enable ensuring they are maintained dry/clean (under a nitrogen blanket). In some examples, the cart may include various processors or electronic controllers configured for programming/monitoring/reporting water status and parameters. Parameters may include oxygen saturation, temperature, particulate debris, pH, mix ratio, flow rate, fill level, power level/battery level, etc., which can be detected in real-time by any number of sensors disposed within and around the system. The parameters may be read out on a UI screen on the fluidics cart, and/or may be displayed/controlled on the therapy system cart display (through software UI).

The degassing module may contain filters or degassing membranes configured to remove particulate/debris, a de-gas contactor and a vacuum or peristaltic pump to move fluid through the system. In some examples, filters may be 0.2 micron in pore size. The de-gas contactor may be able to pull down to parts per billion, with around 3 gallon per minute flow, and capable of removing dissolved O₂, CO₂ and N₂ gas. Vacuum pumps may include key features such as pure transfer and evacuation, high compatibility with vapors and condensation, chemical resistance, and gas tight (very low leakage). In some examples, vacuum pumps are cable of pulling down to 8 torr. In some embodiments, the degassing system can omit the pump and can rely on the water source flow rate (e.g., tap water flow rate) to move the fluid through the system.

The tubing/connectors/lines, plastic and/or metallic, are configured to allow fluid and air communication through the system and overall acoustic/patient coupling system. These may also contain various components such as valves (e.g., two way, three way, etc.).

The electronic and manual controls provide system and user-facing system controls over all the functions of the system, including but not limited to pump and de-gassing controls. The control systems may further comprise various sensors, in-line and onboard, for sensing temperature, pressure, flow rate, dissolved oxygen concentration, volume, etc.

The fluidics system and cart may also have various electrical connections for power including leveraging external power, and/or may comprise a battery/toroid for enabling a detethered fully mobile configuration. This allows the fluidics cart to be wheeled up to prepare/set up a histotripsy procedure, and then wheel away once all fluidics related work-flow steps are complete, so as to not require the fluidics cart to be patient side during treatment/therapy.

The fluidics cart architecture and design may also include handles, individual or central locking casters, a top work surface, embedded user display devices, connectivity (e.g., ethernet, etc.), and may be designed to allow further integration of the support arm in some embodiments. It may also be outfitted with long/extended tubing to support intra-imaging system filling/draining, if for example, use within a CT or MRI, is desirable, so as to not have the overall medium/water volume in close proximity to the scanner, and/or filling during set up is required to further assess image/body divergence pre/post filling.

As for additional details pertinent to the present invention, materials and manufacturing techniques may be employed as within the level of those with skill in the relevant art. The same may hold true with respect to method-based aspects of the invention in terms of additional acts commonly or logically employed. Also, it is contemplated that any optional feature of the inventive variations described may be set forth and claimed independently, or in combination with any one or more of the features described herein. Likewise, reference to a singular item, includes the possibility that there are plural of the same items present. More specifically, as used herein and in the appended claims, the singular forms “a,” “and,” “said,” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs. The breadth of the present invention is not to be limited by the subject specification, but rather only by the plain meaning of the claim terms employed.

What is claimed is:

1. A constraint device, comprising:
a patient section configured to be placed under a patient; and
one or more peripheral sections extending from the patient section configured to support an expandable portion of an ultrasound coupling assembly and including one or more engagement features configured to attach the constraint device to a frame of the ultrasound coupling assembly.

2. The device of claim 1, wherein the first and second peripheral sections include a mesh structure comprising a plurality of openings.

3. The device of claim 1, wherein the constraint device comprises an open pore structure.

4. The device of claim 1, wherein the first and second peripheral sections include a hexagonal pattern comprising a plurality of openings.

5. The device of claim 2, wherein the plurality of openings are configured to engage with one or more attachment features of the rigid portion of the coupling interface.

6. The device of claim 1, wherein the constraint device comprises an elastomeric material.

7. The device of claim 6, wherein a flexibility of the constraint device is less than a flexibility of the expandable portion of the ultrasound coupling assembly.

8. The device of claim 1, at least the first section has a first mesh pattern, and the central section has a second mesh pattern.

9. The device of claim 1, wherein the first mesh pattern is different than the second mesh pattern.

10. The device of claim 1, wherein the first and second sections have a first width proximal to the central section and have a second, larger width distal to the central section.

11. An ultrasound coupling system, comprising:
a coupling container including one or more attachment features;
a coupling membrane coupled to coupling container, the coupling container and coupling membrane forming leak-proof fluidic seal, the coupling membrane being configured to expand to conform to a patient when the coupling container and coupling membrane are filled with an ultrasound coupling medium; and
a porous constraint device being configured to attach to the attachment features of the coupling container, the porous constraint device limiting expansion of the coupling membrane.

12. The ultrasound coupling system of claim 11, wherein the porous constraint device is configured to limit expansion of the coupling membrane when the coupling membrane is filled with the ultrasound coupling medium.

13. The system of claim 11, wherein porous constraint device includes first and second sections having a mesh structure comprising a plurality of openings.

14. The system of claim 11, wherein the porous constraint device comprises an open pore structure.

15. The device of claim 13, wherein the first and second sections include a hexagonal pattern comprising a plurality of openings.

16. The device of claim 11, wherein the porous constraint device comprises an elastomeric material.

17. The device of claim 16, wherein an elasticity of the porous constraint device is less than an elasticity of the coupling membrane.

18. The device of claim 13, wherein the first and second sections have a first mesh pattern, wherein the porous constraint device further comprises a central section between the first and second sections that has a second mesh pattern.

19. The device of claim 17, wherein the first and second sections have a first width proximal to the central section and have a second, larger width distal to the central section.

20. A constraint system for use with a histotripsy system, the constraint system comprising;
an ultrasound coupling assembly comprising a rigid frame having attachment features disposed on an exterior surface therein, and an expandable membrane fixedly attached to the rigid frame; and
a constraint device comprising a central section and one or more peripheral sections extending outward from the central section and including one or more engagement features configured to attach the constraint device to the attachment features.
